SEQUENCE LISTING (141 sequences)

SEQ ID NO: 1 - protein, 4 aa, Artificial Sequence
  Description of Artificial Sequence Synthetic peptide
KKKK

SEQ ID NO: 2 - protein, 8 aa, Artificial Sequence
  Description of Artificial Sequence Synthetic peptide
KKKKKKKK

SEQ ID NO: 3 - protein, 12 aa, Artificial Sequence
  Description of Artificial Sequence Synthetic peptide
KKKKKKKKKKKK

SEQ ID NO: 4 - protein, 16 aa, Artificial Sequence
  Description of Artificial Sequence Synthetic peptide
KKKKKKKKKKKKKKKK

SEQ ID NO: 5 - protein, 4 aa, Artificial Sequence
  Description of Artificial Sequence Synthetic peptide
RRRR

SEQ ID NO: 6 - protein, 8 aa, Artificial Sequence
  Description of Artificial Sequence Synthetic peptide
RRRRRRRR

SEQ ID NO: 7 - protein, 12 aa, Artificial Sequence
  Description of Artificial Sequence Synthetic peptide
RRRRRRRRRRRR

SEQ ID NO: 8 - protein, 16 aa, Artificial Sequence
  Description of Artificial Sequence Synthetic peptide
RRRRRRRRRRRRRRRR

SEQ ID NO: 9 - protein, 4 aa, Artificial Sequence
  Description of Artificial Sequence Synthetic peptide
KRKR

SEQ ID NO: 10 - protein, 4 aa, Artificial Sequence
  Description of Artificial Sequence Synthetic peptide
KKKR

SEQ ID NO: 11 - protein, 12 aa, Artificial Sequence
  Description of Artificial Sequence Synthetic peptide
KKKRRRKKKRRR

SEQ ID NO: 12 - protein, 16 aa, Artificial Sequence
  Description of Artificial Sequence Synthetic peptide
KKKKRRRRKKKKRRRR

SEQ ID NO: 13 - protein, 20 aa, Artificial Sequence
  Description of Artificial Sequence Synthetic peptide
LRVRLASHLRKLRKRLLRDA

SEQ ID NO: 14 - protein, 40 aa, Artificial Sequence
  Description of Artificial Sequence Synthetic polypeptide
SSVIDALQYKLEGTTRLTRKRGLKLATALSLSNKFVEGSH

SEQ ID NO: 15 - protein, 36 aa, Artificial Sequence
  Description of Artificial Sequence Synthetic polypeptide
YPAKPEAPGEDASPEELSRYYASLRHYLNLVTRQRX

SEQ ID NO: 16 - protein, 34 aa, Artificial Sequence
  Description of Artificial Sequence Synthetic polypeptide
AKPEAPGEDASPEELSRYYASLRHYLNLVTRQRX

SEQ ID NO: 17 - protein, 36 aa, Artificial Sequence
  Description of Artificial Sequence Synthetic polypeptide
YPSDPDNPGEDAPAEDLARYYSALRHYINLITRQRX

SEQ ID NO: 18 - protein, 36 aa, Artificial Sequence
  Description of Artificial Sequence Synthetic polypeptide
APLEPVYPGDDATPEQMAQYAAELRRYINMLTRPRX

SEQ ID NO: 19 - protein, 20 aa, Artificial Sequence
  Description of Artificial Sequence Synthetic peptide
LRKLRKRLLRLRKLRKRLLR

SEQ ID NO: 20 - protein, 20 aa, Artificial Sequence
  Description of Artificial Sequence Synthetic peptide
LRXRXXXHLRXXXKRLXRDX

SEQ ID NO: 21 - protein, 20 aa, Artificial Sequence
  Description of Artificial Sequence Synthetic peptide
LRSRLASHLRKLRKRLLRDA

SEQ ID NO: 22 - protein, 20 aa, Artificial Sequence
  Description of Artificial Sequence Synthetic peptide
LRVRMASHLRKLRKRLLRDA

SEQ ID NO: 23 - protein, 20 aa, Artificial Sequence
  Description of Artificial Sequence Synthetic peptide
LRVRLATHLRKLRKRLLRDA

SEQ ID NO: 24 - protein, 20 aa, Artificial Sequence
  Description of Artificial Sequence Synthetic peptide
LRVRLASHLRKLPKRLLRDA

SEQ ID NO: 25 - protein, 20 aa, Artificial Sequence
  Description of Artificial Sequence Synthetic peptide
LRVRLASHLRKLRKRLMRDA

SEQ ID NO: 26 - protein, 20 aa, Artificial Sequence
  Description of Artificial Sequence Synthetic peptide
LRVRLASHLRNLRKRLLRDA

SEQ ID NO: 27 - protein, 20 aa, Artificial Sequence
  Description of Artificial Sequence Synthetic peptide
LRVRLASHLRKVRKRLLRDA

SEQ ID NO: 28 - protein, 20 aa, Artificial Sequence
  Description of Artificial Sequence Synthetic peptide
LRVRMSSHLRKLRKRLLRDA

SEQ ID NO: 29 - protein, 20 aa, Artificial Sequence
  Description of Artificial Sequence Synthetic peptide
LRVRLASHLRNVRKRLLRDA

SEQ ID NO: 30 - protein, 20 aa, Artificial Sequence
  Description of Artificial Sequence Synthetic peptide
LRVRLASHLRNMRKRLLRDA

SEQ ID NO: 31 - protein, 20 aa, Artificial Sequence
  Description of Artificial Sequence Synthetic peptide
LRARMASHLRKLRKRLLRDA

SEQ ID NO: 32 - protein, 20 aa, Artificial Sequence
  Description of Artificial Sequence Synthetic peptide
LRVRLSSHLRKLRKRLMRDA

SEQ ID NO: 33 - protein, 20 aa, Artificial Sequence
  Description of Artificial Sequence Synthetic peptide
LRSRLASHLRKLRKRLMRDA

SEQ ID NO: 34 - protein, 20 aa, Artificial Sequence
  Description of Artificial Sequence Synthetic peptide
LRVRLSSHLPKLRKRLLRDA

SEQ ID NO: 35 - protein, 20 aa, Artificial Sequence
  Description of Artificial Sequence Synthetic peptide
LRVRLASHLRKMRKRLMRDA

SEQ ID NO: 36 - protein, 20 aa, Artificial Sequence
  Description of Artificial Sequence Synthetic peptide
LRVRLASHLRNLPKRLLRDA

SEQ ID NO: 37 - protein, 20 aa, Artificial Sequence
  Description of Artificial Sequence Synthetic peptide
LRLRLASHLRKLRKRLLRDL

SEQ ID NO: 38 - protein, 20 aa, Artificial Sequence
  Description of Artificial Sequence Synthetic peptide
LRVRLANHLRKLRKRLLRDL

SEQ ID NO: 39 - protein, 24 aa, Artificial Sequence
  Description of Artificial Sequence Synthetic peptide
KKKKLRVRLASHLRKLRKRLLRDA

SEQ ID NO: 40 - protein, 28 aa, Artificial Sequence
  Description of Artificial Sequence Synthetic peptide
KKKKKKKKLRVRLASHLRKLRKRLLRDA

SEQ ID NO: 41 - protein, 32 aa, Artificial Sequence
  Description of Artificial Sequence Synthetic polypeptide
KKKKKKKKKKKKLRVRLASHLRKLRKRLLRDA

SEQ ID NO: 42 - protein, 36 aa, Artificial Sequence
  Description of Artificial Sequence Synthetic polypeptide
KKKKKKKKKKKKKKKKLRVRLASHLRKLRKRLLRDA

SEQ ID NO: 43 - protein, 40 aa, Artificial Sequence
  Description of Artificial Sequence Synthetic polypeptide
KKKKKKKKKKKKKKKKKKKKLRVRLASHLRKLRKRLLRDA

SEQ ID NO: 44 - protein, 44 aa, Artificial Sequence
  Description of Artificial Sequence Synthetic polypeptide
KKKKSSVIDALQYKLEGTTRLTRKRGLKLATALSLSNKFVEGSH

SEQ ID NO: 45 - protein, 48 aa, Artificial Sequence
  Description of Artificial Sequence Synthetic polypeptide
KKKKKKKKSSVIDALQYKLEGTTRLTRKRGLKLATALSLSNKFVEGSH

SEQ ID NO: 46 - protein, 52 aa, Artificial Sequence
  Description of Artificial Sequence Synthetic polypeptide
KKKKKKKKKKKKSSVIDALQYKLEGTTRLTRKRGLKLATALSLSNKFVEGSH

SEQ ID NO: 47 - protein, 56 aa, Artificial Sequence
  Description of Artificial Sequence Synthetic polypeptide
KKKKKKKKKKKKKKKKSSVIDALQYKLEGTTRLTRKRGLKLATALSLSNKFVEGSH

SEQ ID NO: 48 - protein, 60 aa, Artificial Sequence
  Description of Artificial Sequence Synthetic polypeptide
KKKKKKKKKKKKKKKKKKKKSSVIDALQYKLEGTTRLTRKRGLKLATALSLSNKFVEGSH

SEQ ID NO: 49 - protein, 24 aa, Artificial Sequence
  Description of Artificial Sequence Synthetic peptide
KKKKLRSRLASHLRKLRKRLLRDA

SEQ ID NO: 50 - protein, 28 aa, Artificial Sequence
  Description of Artificial Sequence Synthetic peptide
KKKKKKKKLRSRLASHLRKLRKRLLRDA

SEQ ID NO: 51 - protein, 32 aa, Artificial Sequence
  Description of Artificial Sequence Synthetic polypeptide
KKKKKKKKKKKKLRSRLASHLRKLRKRLLRDA

SEQ ID NO: 52 - protein, 36 aa, Artificial Sequence
  Description of Artificial Sequence Synthetic polypeptide
KKKKKKKKKKKKKKKKLRSRLASHLRKLRKRLLRDA

SEQ ID NO: 53 - protein, 40 aa, Artificial Sequence
  Description of Artificial Sequence Synthetic polypeptide
KKKKKKKKKKKKKKKKKKKKLRSRLASHLRKLRKRLLRDA

SEQ ID NO: 54 - protein, 24 aa, Artificial Sequence
  Description of Artificial Sequence Synthetic peptide
KKKKLRVRMASHLRKLRKRLLRDA

SEQ ID NO: 55 - protein, 28 aa, Artificial Sequence
  Description of Artificial Sequence Synthetic peptide
KKKKKKKKLRVRMASHLRKLRKRLLRDA

SEQ ID NO: 56 - protein, 32 aa, Artificial Sequence
  Description of Artificial Sequence Synthetic polypeptide
KKKKKKKKKKKKLRVRMASHLRKLRKRLLRDA

SEQ ID NO: 57 - protein, 36 aa, Artificial Sequence
  Description of Artificial Sequence Synthetic polypeptide
KKKKKKKKKKKKKKKKLRVRMASHLRKLRKRLLRDA

SEQ ID NO: 58 - protein, 40 aa, Artificial Sequence
  Description of Artificial Sequence Synthetic polypeptide
KKKKKKKKKKKKKKKKKKKKLRVRMASHLRKLRKRLLRDA

SEQ ID NO: 59 - protein, 24 aa, Artificial Sequence
  Description of Artificial Sequence Synthetic peptide
KKKKLRVRLATHLRKLRKRLLRDA

SEQ ID NO: 60 - protein, 28 aa, Artificial Sequence
  Description of Artificial Sequence Synthetic peptide
KKKKKKKKLRVRLATHLRKLRKRLLRDA

SEQ ID NO: 61 - protein, 32 aa, Artificial Sequence
  Description of Artificial Sequence Synthetic polypeptide
KKKKKKKKKKKKLRVRLATHLRKLRKRLLRDA

SEQ ID NO: 62 - protein, 36 aa, Artificial Sequence
  Description of Artificial Sequence Synthetic polypeptide
KKKKKKKKKKKKKKKKLRVRLATHLRKLRKRLLRDA

SEQ ID NO: 63 - protein, 40 aa, Artificial Sequence
  Description of Artificial Sequence Synthetic polypeptide
KKKKKKKKKKKKKKKKKKKKLRVRLATHLRKLRKRLLRDA

SEQ ID NO: 64 - protein, 24 aa, Artificial Sequence
  Description of Artificial Sequence Synthetic peptide
KKKKLRVRLASHLRKLPKRLLRDA

SEQ ID NO: 65 - protein, 28 aa, Artificial Sequence
  Description of Artificial Sequence Synthetic peptide
KKKKKKKKLRVRLASHLRKLPKRLLRDA

SEQ ID NO: 66 - protein, 32 aa, Artificial Sequence
  Description of Artificial Sequence Synthetic polypeptide
KKKKKKKKKKKKLRVRLASHLRKLPKRLLRDA

SEQ ID NO: 67 - protein, 36 aa, Artificial Sequence
  Description of Artificial Sequence Synthetic polypeptide
KKKKKKKKKKKKKKKKLRVRLASHLRKLPKRLLRDA

SEQ ID NO: 68 - protein, 40 aa, Artificial Sequence
  Description of Artificial Sequence Synthetic polypeptide
KKKKKKKKKKKKKKKKKKKKLRVRLASHLRKLPKRLLRDA

SEQ ID NO: 69 - protein, 24 aa, Artificial Sequence
  Description of Artificial Sequence Synthetic peptide
KKKKLRVRLASHLRKLRKRLMRDA

SEQ ID NO: 70 - protein, 28 aa, Artificial Sequence
  Description of Artificial Sequence Synthetic peptide
KKKKKKKKLRVRLASHLRKLRKRLMRDA

SEQ ID NO: 71 - protein, 32 aa, Artificial Sequence
  Description of Artificial Sequence Synthetic polypeptide
KKKKKKKKKKKKLRVRLASHLRKLRKRLMRDA

SEQ ID NO: 72 - protein, 36 aa, Artificial Sequence
  Description of Artificial Sequence Synthetic polypeptide
KKKKKKKKKKKKKKKKLRVRLASHLRKLRKRLMRDA

SEQ ID NO: 73 - protein, 40 aa, Artificial Sequence
  Description of Artificial Sequence Synthetic polypeptide
KKKKKKKKKKKKKKKKKKKKLRVRLASHLRKLRKRLMRDA

SEQ ID NO: 74 - protein, 24 aa, Artificial Sequence
  Description of Artificial Sequence Synthetic peptide
KKKKLRVRLASHLRNLRKRLLRDA

SEQ ID NO: 75 - protein, 28 aa, Artificial Sequence
  Description of Artificial Sequence Synthetic peptide
KKKKKKKKLRVRLASHLRNLRKRLLRDA

SEQ ID NO: 76 - protein, 32 aa, Artificial Sequence
  Description of Artificial Sequence Synthetic polypeptide
KKKKKKKKKKKKLRVRLASHLRNLRKRLLRDA

SEQ ID NO: 77 - protein, 36 aa, Artificial Sequence
  Description of Artificial Sequence Synthetic polypeptide
KKKKKKKKKKKKKKKKLRVRLASHLRNLRKRLLRDA

SEQ ID NO: 78 - protein, 40 aa, Artificial Sequence
  Description of Artificial Sequence Synthetic polypeptide
KKKKKKKKKKKKKKKKKKKKLRVRLASHLRNLRKRLLRDA

SEQ ID NO: 79 - protein, 24 aa, Artificial Sequence
  Description of Artificial Sequence Synthetic peptide
KKKKLRVRLASHLRKVRKRLLRDA

SEQ ID NO: 80 - protein, 28 aa, Artificial Sequence
  Description of Artificial Sequence Synthetic peptide
KKKKKKKKLRVRLASHLRKVRKRLLRDA

SEQ ID NO: 81 - protein, 32 aa, Artificial Sequence
  Description of Artificial Sequence Synthetic polypeptide
KKKKKKKKKKKKLRVRLASHLRKVRKRLLRDA

SEQ ID NO: 82 - protein, 36 aa, Artificial Sequence
  Description of Artificial Sequence Synthetic polypeptide
KKKKKKKKKKKKKKKKLRVRLASHLRKVRKRLLRDA

SEQ ID NO: 83 - protein, 40 aa, Artificial Sequence
  Description of Artificial Sequence Synthetic polypeptide
KKKKKKKKKKKKKKKKKKKKLRVRLASHLRKVRKRLLRDA

SEQ ID NO: 84 - protein, 24 aa, Artificial Sequence
  Description of Artificial Sequence Synthetic peptide
KKKKLRVRMSSHLRKLRKRLLRDA

SEQ ID NO: 85 - protein, 28 aa, Artificial Sequence
  Description of Artificial Sequence Synthetic peptide
KKKKKKKKLRVRMSSHLRKLRKRLLRDA

SEQ ID NO: 86 - protein, 32 aa, Artificial Sequence
  Description of Artificial Sequence Synthetic polypeptide
KKKKKKKKKKKKLRVRMSSHLRKLRKRLLRDA

SEQ ID NO: 87 - protein, 36 aa, Artificial Sequence
  Description of Artificial Sequence Synthetic polypeptide
KKKKKKKKKKKKKKKKLRVRMSSHLRKLRKRLLRDA

SEQ ID NO: 88 - protein, 40 aa, Artificial Sequence
  Description of Artificial Sequence Synthetic polypeptide
KKKKKKKKKKKKKKKKKKKKLRVRMSSHLRKLRKRLLRDA

SEQ ID NO: 89 - protein, 24 aa, Artificial Sequence
  Description of Artificial Sequence Synthetic peptide
KKKKLRVRLASHLRNVRKRLLRDA

SEQ ID NO: 90 - protein, 28 aa, Artificial Sequence
  Description of Artificial Sequence Synthetic peptide
KKKKKKKKLRVRLASHLRNVRKRLLRDA

SEQ ID NO: 91 - protein, 32 aa, Artificial Sequence
  Description of Artificial Sequence Synthetic polypeptide
KKKKKKKKKKKKLRVRLASHLRNVRKRLLRDA

SEQ ID NO: 92 - protein, 36 aa, Artificial Sequence
  Description of Artificial Sequence Synthetic polypeptide
KKKKKKKKKKKKKKKKLRVRLASHLRNVRKRLLRDA

SEQ ID NO: 93 - protein, 40 aa, Artificial Sequence
  Description of Artificial Sequence Synthetic polypeptide
KKKKKKKKKKKKKKKKKKKKLRVRLASHLRNVRKRLLRDA

SEQ ID NO: 94 - protein, 24 aa, Artificial Sequence
  Description of Artificial Sequence Synthetic peptide
KKKKLRVRLASHLRNMRKRLLRDA

SEQ ID NO: 95 - protein, 28 aa, Artificial Sequence
  Description of Artificial Sequence Synthetic peptide
KKKKKKKKLRVRLASHLRNMRKRLLRDA

SEQ ID NO: 96 - protein, 32 aa, Artificial Sequence
  Description of Artificial Sequence Synthetic polypeptide
KKKKKKKKKKKKLRVRLASHLRNMRKRLLRDA

SEQ ID NO: 97 - protein, 36 aa, Artificial Sequence
  Description of Artificial Sequence Synthetic polypeptide
KKKKKKKKKKKKKKKKLRVRLASHLRNMRKRLLRDA

SEQ ID NO: 98 - protein, 40 aa, Artificial Sequence
  Description of Artificial Sequence Synthetic polypeptide
KKKKKKKKKKKKKKKKKKKKLRVRLASHLRNMRKRLLRDA

SEQ ID NO: 99 - protein, 24 aa, Artificial Sequence
  Description of Artificial Sequence Synthetic peptide
KKKKLRARMASHLRKLRKRLLRDA

SEQ ID NO: 100 - protein, 28 aa, Artificial Sequence
  Description of Artificial Sequence Synthetic peptide
KKKKKKKKLRARMASHLRKLRKRLLRDA

SEQ ID NO: 101 - protein, 32 aa, Artificial Sequence
  Description of Artificial Sequence Synthetic polypeptide
KKKKKKKKKKKKLRARMASHLRKLRKRLLRDA

SEQ ID NO: 102 - protein, 36 aa, Artificial Sequence
  Description of Artificial Sequence Synthetic polypeptide
KKKKKKKKKKKKKKKKLRARMASHLRKLRKRLLRDA

SEQ ID NO: 103 - protein, 40 aa, Artificial Sequence
  Description of Artificial Sequence Synthetic polypeptide
KKKKKKKKKKKKKKKKKKKKLRARMASHLRKLRKRLLRDA

SEQ ID NO: 104 - protein, 24 aa, Artificial Sequence
  Description of Artificial Sequence Synthetic peptide
KKKKLRVRLSSHLRKLRKRLMRDA

SEQ ID NO: 105 - protein, 28 aa, Artificial Sequence
  Description of Artificial Sequence Synthetic peptide
KKKKKKKKLRVRLSSHLRKLRKRLMRDA

SEQ ID NO: 106 - protein, 32 aa, Artificial Sequence
  Description of Artificial Sequence Synthetic polypeptide
KKKKKKKKKKKKLRVRLSSHLRKLRKRLMRDA

SEQ ID NO: 107 - protein, 36 aa, Artificial Sequence
  Description of Artificial Sequence Synthetic polypeptide
KKKKKKKKKKKKKKKKLRVRLSSHLRKLRKRLMRDA

SEQ ID NO: 108 - protein, 40 aa, Artificial Sequence
  Description of Artificial Sequence Synthetic polypeptide
KKKKKKKKKKKKKKKKKKKKLRVRLSSHLRKLRKRLMRDA

SEQ ID NO: 109 - protein, 24 aa, Artificial Sequence
  Description of Artificial Sequence Synthetic peptide
KKKKLRSRLASHLRKLRKRLMRDA

SEQ ID NO: 110 - protein, 28 aa, Artificial Sequence
  Description of Artificial Sequence Synthetic peptide
KKKKKKKKLRSRLASHLRKLRKRLMRDA

SEQ ID NO: 111 - protein, 32 aa, Artificial Sequence
  Description of Artificial Sequence Synthetic polypeptide
KKKKKKKKKKKKLRSRLASHLRKLRKRLMRDA

SEQ ID NO: 112 - protein, 36 aa, Artificial Sequence
  Description of Artificial Sequence Synthetic polypeptide
KKKKKKKKKKKKKKKKLRSRLASHLRKLRKRLMRDA

SEQ ID NO: 113 - protein, 40 aa, Artificial Sequence
  Description of Artificial Sequence Synthetic polypeptide
KKKKKKKKKKKKKKKKKKKKLRSRLASHLRKLRKRLMRDA

SEQ ID NO: 114 - protein, 24 aa, Artificial Sequence
  Description of Artificial Sequence Synthetic peptide
KKKKLRVRLSSHLPKLRKRLLRDA

SEQ ID NO: 115 - protein, 28 aa, Artificial Sequence
  Description of Artificial Sequence Synthetic peptide
KKKKKKKKLRVRLSSHLPKLRKRLLRDA

SEQ ID NO: 116 - protein, 32 aa, Artificial Sequence
  Description of Artificial Sequence Synthetic polypeptide
KKKKKKKKKKKKLRVRLSSHLPKLRKRLLRDA

SEQ ID NO: 117 - protein, 36 aa, Artificial Sequence
  Description of Artificial Sequence Synthetic polypeptide
KKKKKKKKKKKKKKKKLRVRLSSHLPKLRKRLLRDA

SEQ ID NO: 118 - protein, 40 aa, Artificial Sequence
  Description of Artificial Sequence Synthetic polypeptide
KKKKKKKKKKKKKKKKKKKKLRVRLSSHLPKLRKRLLRDA

SEQ ID NO: 119 - protein, 24 aa, Artificial Sequence
  Description of Artificial Sequence Synthetic peptide
KKKKLRVRLASHLRKMRKRLMRDA

SEQ ID NO: 120 - protein, 28 aa, Artificial Sequence
  Description of Artificial Sequence Synthetic peptide
KKKKKKKKLRVRLASHLRKMRKRLMRDA

SEQ ID NO: 121 - protein, 32 aa, Artificial Sequence
  Description of Artificial Sequence Synthetic polypeptide
KKKKKKKKKKKKLRVRLASHLRKMRKRLMRDA

SEQ ID NO: 122 - protein, 36 aa, Artificial Sequence
  Description of Artificial Sequence Synthetic polypeptide
KKKKKKKKKKKKKKKKLRVRLASHLRKMRKRLMRDA

SEQ ID NO: 123 - protein, 40 aa, Artificial Sequence
  Description of Artificial Sequence Synthetic polypeptide
KKKKKKKKKKKKKKKKKKKKLRVRLASHLRKMRKRLMRDA

SEQ ID NO: 124 - protein, 24 aa, Artificial Sequence
  Description of Artificial Sequence Synthetic peptide
KKKKLRVRLASHLRNLPKRLLRDA

SEQ ID NO: 125 - protein, 28 aa, Artificial Sequence
  Description of Artificial Sequence Synthetic peptide
KKKKKKKKLRVRLASHLRNLPKRLLRDA

SEQ ID NO: 126 - protein, 32 aa, Artificial Sequence
  Description of Artificial Sequence Synthetic polypeptide
KKKKKKKKKKKKLRVRLASHLRNLPKRLLRDA

SEQ ID NO: 127 - protein, 36 aa, Artificial Sequence
  Description of Artificial Sequence Synthetic polypeptide
KKKKKKKKKKKKKKKKLRVRLASHLRNLPKRLLRDA

SEQ ID NO: 128 - protein, 40 aa, Artificial Sequence
  Description of Artificial Sequence Synthetic polypeptide
KKKKKKKKKKKKKKKKKKKKLRVRLASHLRNLPKRLLRDA

SEQ ID NO: 129 - protein, 24 aa, Artificial Sequence
  Description of Artificial Sequence Synthetic peptide
KKKKLRLRLASHLRKLRKRLLRDL

SEQ ID NO: 130 - protein, 28 aa, Artificial Sequence
  Description of Artificial Sequence Synthetic peptide
KKKKKKKKLRLRLASHLRKLRKRLLRDL

SEQ ID NO: 131 - protein, 32 aa, Artificial Sequence
  Description of Artificial Sequence Synthetic polypeptide
KKKKKKKKKKKKLRLRLASHLRKLRKRLLRDL

SEQ ID NO: 132 - protein, 36 aa, Artificial Sequence
  Description of Artificial Sequence Synthetic polypeptide
KKKKKKKKKKKKKKKKLRLRLASHLRKLRKRLLRDL

SEQ ID NO: 133 - protein, 40 aa, Artificial Sequence
  Description of Artificial Sequence Synthetic polypeptide
KKKKKKKKKKKKKKKKKKKKLRLRLASHLRKLRKRLLRDL

SEQ ID NO: 134 - protein, 24 aa, Artificial Sequence
  Description of Artificial Sequence Synthetic peptide
KKKKLRVRLANHLRKLRKRLLRDL

SEQ ID NO: 135 - protein, 28 aa, Artificial Sequence
  Description of Artificial Sequence Synthetic peptide
KKKKKKKKLRVRLANHLRKLRKRLLRDL

SEQ ID NO: 136 - protein, 32 aa, Artificial Sequence
  Description of Artificial Sequence Synthetic polypeptide
KKKKKKKKKKKKLRVRLANHLRKLRKRLLRDL

SEQ ID NO: 137 - protein, 36 aa, Artificial Sequence
  Description of Artificial Sequence Synthetic polypeptide
KKKKKKKKKKKKKKKKLRVRLANHLRKLRKRLLRDL

SEQ ID NO: 138 - protein, 40 aa, Artificial Sequence
  Description of Artificial Sequence Synthetic polypeptide
KKKKKKKKKKKKKKKKKKKKLRVRLANHLRKLRKRLLRDL

SEQ ID NO: 139 - protein, 46 aa, Artificial Sequence
  Description of Artificial Sequence Synthetic polypeptide
KKKKKKKKKKKKKKKKKKKKKKKKKKLRVRLASHLRKLRKRLLRDA

SEQ ID NO: 140 - protein, 40 aa, Artificial Sequence
  Description of Artificial Sequence Synthetic polypeptide
XXXXXXXXXXXXXXXXXXXXLRXRXXXHLRXXXKRLXRDX

SEQ ID NO: 141 - protein, 70 aa, Artificial Sequence
  Description of Artificial Sequence Synthetic polypeptide
XXXXXXXXXXXXXXXXXXXXXXXXXXXXXXXXXXXXXXXXXXXXXXXXXXLRXRXXXHLRXXXKRLXRDX

(19) United States
(12) Patent Application Publication
(10) Pub. No.: US 2014/0314663 A1
(43) Pub. Date: Oct. 23, 2014

(54) PEPTIDE-MEDIATED DELIVERY OF ACTIVE AGENTS ACROSS THE BLOOD-BRAIN BARRIER

(71) Applicant: MAYO FOUNDATION FOR MEDICAL EDUCATION AND RESEARCH, Robertes, MN (US)

(72) Inventors: Gobinda Sarkar, Rochester, MN (US); Geoffry L. Curran, Rochester, MN (US); Robert B. Jenkins, Rochester, MN (US)

(73) Assignee: MAYO FOUNDATION FOR MEDICAL EDUCATION AND RESEARCH, Rochester, MN (US)

(21) Appl. No.: 14/361,190

(22) PCT Filed: Nov. 29, 2012
(86) PCT No.: PCT/US2012/067065

§ 371 Date: May 28, 2014

Related U.S. Application Data
(60) Provisional application No. 61/564,397, filed on Nov. 29, 2011.

(51) Int. Cl. A61K 47/42; A61K 38/17; A61K 33/24; A61K 49/00

(52) U.S. Cl. CPC A61K 47/42; A61K 49/0004; A61K 38/1709; A61K 33/24
USPC 424/1.11; 514/773; 424/9.1; 514/1.1; 514/44 R; 424/172.1; 514/54; 514/23; 424/184.1; 514/44 A

(57) ABSTRACT

Provided herein are methods for transporting an active agent (e.g., a therapeutic agent or an imaging agent) across the blood-brain barrier (BBB) in a patient. For example, the methods provided herein can be used to treat a brain disease in a patient, such as brain cancer. In some embodiments, the methods provided herein can be used to image the central nervous system of a patient.

### CROSS-REFERENCE TO RELATED APPLICATIONS

This application claims the benefit of U.S. Provisional Application Ser. No. 61/564,397, filed Nov. 29, 2011. The disclosure of the prior applications are considered part of (and is incorporated by reference in) the disclosure of this application.

### SEQUENCE LISTING

The instant application contains a Sequence Listing which has been submitted in ASCII format via EFS-Web and is hereby incorporated by reference in its entirety. Said ASCII copy, created on Nov. 29, 2012, is named Sequence_listing.txt and is 59,614 bytes in size.

### TECHNICAL FIELD

This disclosure relates to methods of delivering active agents across the blood-brain barrier using synthetic peptides.

### BACKGROUND

The blood-brain barrier (BBB) prevents most macromolecules (e.g., DNA, RNA, and polypeptides) and many small molecules from entering the brain. The BBB is principally composed of specialized endothelial cells with highly restrictive tight junctions, consequently, passage of substances, small and large, from the blood into the central nervous system is controlled by the BBB. This structure makes treatment and management of patients with neurological diseases and disorders (e.g., brain cancer and Alzheimer's disease) difficult as many therapeutic agents cannot be delivered across the BBB with desirable efficiency.

### SUMMARY

Provided herein are methods for transporting an active agent (e.g., a therapeutic agent or an imaging agent) across the blood-brain barrier (BBB) in a patient. For example, the methods provided herein can be used to treat a brain disease in a patient, such as brain cancer. In some embodiments, the methods provided herein can be used to image the central nervous system of a patient.

The methods provided herein provide several advantages over other methods of transporting agents across the BBB. Several strategies have been undertaken to overcome the restriction imposed by the BBB for delivering therapeutic agents into the brain. In many cases, these approaches involve physical and/or chemical means to transiently open the BBB for subsequent passage of therapeutics (especially small molecules) across the barrier. Many of these methods have limitations. For example, convection-enhanced delivery requires some invasive procedures (introduction of needles and catheters) and results in ineffective tissue distribution of the drugs injected; intra-arterial injection of hyperosmolar agents such as mannitol causes temporal and reversible disruption of the BBB for delivering drugs but the strategy is believed to cause diffusive disruption of the BBB and may cause significant expansion of the vascular volume. Drug delivery across the BBB by ultrasound is another means, which is currently being investigated in several laboratories. The method appears to have some limitations since it basically works by means of creating microbubbles, the size of which is difficult to control. The method is also believed to cause irreversible damage to blood vessels, and can also damage endothelial cells. Since lipid solubility enhances passive diffusion of a molecule across the BBB, such chemical modification (lipidization) to deliver drugs to the brain has also been used. This approach also has limitations since chemical modification of a drug (lipidization) is an expensive and time-consuming process, and also because the process may alter the pharmacokinetic properties of the drug.

Provided herein are synthetic peptides (e.g., synthetic proteins including apolipoprotein E (ApoE) such as K16ApoE (SEQ ID NO: 42) which are believed to bind with various proteins (e.g., albumin and/or immunoglobulins) and assume an ApoE-like structure which is presumably recognized by the low-density lipoprotein receptor (LDLR) pathway at the BBB, allowing passage across the BBB through transcytosis. Without being bound by theory, it is believed that since the synthetic peptide is not a native BBB peptide, it causes a conformational change of the BBB creating transient pores at the barrier, which subsequently allows transport of active agents (e.g., small molecules) to the brain.

Provided herein is a method of transporting an active agent across the blood-brain barrier of a patient, the method comprising:
(a) first administering to the patient an effective amount of a peptide comprising the sequence:

Xₙ-[B]ₘ

wherein:
X is a hydrophilic amino acid;
B is a blood-brain barrier agent;
n is an integer from 4 to 50; and
m is an integer from 1 to 3; and
(b) then administering to the patient an effective amount of the active agent.

Also provided herein is a method of treating a brain disease in a patient, the method comprising:
(a) first administering to the patient an effective amount of a peptide comprising the sequence:

Xₙ-[B]ₘ

wherein:
X is a hydrophilic amino acid;
B is a blood-brain barrier agent;
n is an integer from 4 to 50; and
m is an integer from 1 to 3; and
(b) then administering to the patient a therapeutically effective amount of a biologically active agent for treatment of the brain disease.

Further provided herein is a method of imaging the central-nervous system in a patient, the method comprising:
(a) first administering to the patient an effective amount of a peptide comprising the sequence:

Xₙ-[B]ₘ

wherein:
X is a hydrophilic amino acid;
B is a blood-brain barrier agent;
n is an integer from 4 to 50; and
m is an integer from 1 to 3; and
(b) then administering to the patient an effective amount of an imaging agent; and
(c) imaging the central-nervous system of the patient.

In some embodiments of the methods provided herein, X is independently selected from the group consisting of arginine, asparagine, aspartic acid, glutamic acid, glutamine, histidine, lysine, serine, threonine, and tyrosine. For example, each X can be lysine.

The variable n can be selected from the group consisting of: 4, 8, 12, 16, and 20. In some embodiments, n is 16.

In some embodiments, m is 1.

In some embodiments, B is a receptor binding domain of an apolipoprotein. For example, the receptor binding domain of an apolipoprotein is chosen from the receptor binding domain of ApoA, ApoB, ApoC, ApoD, ApoE, ApoE2, ApoE3, and ApoE4. In some embodiments, the receptor binding domain of an apolipoprotein is chosen from the receptor binding domain of ApoB and ApoE.

An active agent can be a biologically active molecule or an imaging agent. For example, a biologically active molecule can be selected from the group consisting of a: polypeptide; oligonucleotide; plasmid; small molecule; antibody; antibody fragment; carbohydrate; polysaccharide; lipid; glycolipid; antigen; and antigenic peptide. In some embodiments, the biologically active molecule is chosen from a: polypeptide; oligonucleotide; and plasmid. For example, an oligonucleotide can be chosen from a: coding DNA sequence; antisense DNA sequence; mRNA, antisense RNA sequence; RNAi; and siRNA. In some embodiments, a small molecule is a therapeutic agent. In some embodiments, an imaging agent comprises one or more of a: radionuclide, a paramagnetic metal, a fluorochrome, a dye, and an enzyme substrate.

A brain disorder can be selected from the group consisting of: meningitis, epilepsy, multiple sclerosis, neuromyelitis optica, late-stage neurological trypanosomiasis, Parkinson's, progressive multifocal leukoencephalopathy, De Vivo disease, Alzheimer's disease, HIV Encephalitis, and cancer.

In some embodiments, an active agent is administered about 5 minutes to about 2 hours after a peptide.

In some embodiments, a peptide is administered in combination with a natural polypeptide capable of binding to a receptor at the blood-brain barrier. For example, a peptide can be administered in combination with IgG.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 shows the results of transportation of Evan's Blue (EB) across the BBB using various peptides and administration techniques.

FIG. 2 illustrates the transportation of EB to the brain as a function of different amounts of K16ApoE (SEQ ID NO: 42) injected.

FIG. 3 shows the results of transportation of various dyes of differing molecular weights across the BBB following iv injection of K16ApoE (SEQ ID NO: 42) and a mixture of K16ApoE (SEQ ID NO: 42) and IgG.

FIG. 4 illustrates how long the BBB remains open following administration of K16ApoE (SEQ ID NO: 42).

FIG. 5 shows the residence time of EB in the brain following transportation across the BBB.

FIG. 6 illustrates the comparison of delivery of EB by K16ApoE (SEQ ID NO: 42) and K26ApoE (SEQ ID NO: 139).

FIG. 7 shows the tissue distribution of EB after delivery via K16ApoE in the brains, lungs, heart, liver, spleen, and kidney of mice.

### DETAILED DESCRIPTION

Provided herein are methods for transporting an active agent (e.g., a therapeutic agent or an imaging agent) across the blood-brain barrier (BBB) in a patient. For example, the methods provided herein can be used to treat a brain disease in a patient, such as brain cancer. In some embodiments, the methods provided herein can be used to image the central nervous system of a patient.

### Peptides

Provided herein are peptides having the following sequence:

Xₙ-[B]ₘ

wherein:
X is a hydrophilic amino acid;
B is a blood-brain barrier agent;
n is an integer from 4 to 50; and
m is integer from 1 to 3.

A hydrophilic amino acid can be chosen from: arginine, asparagine, aspartic acid, glutamic acid, glutamine, histidine, lysine, serine, threonine, tyrosine, and combinations and non-natural derivatives thereof. In some embodiments, a hydrophilic amino acid can be chosen from lysine or a non-natural lysine derivative, arginine or a non-natural arginine derivative, and combinations thereof. In some embodiments, the hydrophilic amino acid is lysine. Non-limiting examples of Xₙ can include KKKK (SEQ ID NO:1); KKKKKKKK (SEQ ID NO:2); KKKKKKKKKKKK (SEQ ID NO:3); (SEQ ID NO:4); RRRR (SEQ ID NO:5); RRRRRRRR (SEQ ID NO:6); RRRRRRRRRRRR (SEQ ID NO:7); RRRRRRRRRRRRRRRR (SEQ ID NO:8); KRKR (SEQ ID NO:9); KKKR (SEQ ID NO:10); KKKRRRKKKRRR (SEQ ID NO:11); and KKKKRRRRKKKKRRRR (SEQ ID NO:12).

The variable n is an integer ranging from 4 to 50 (e.g., 4, 6, 8, 10, 12, 16, 20, 24, 26, 28, 32, 36, 40, 42, 44, 48, and 50). For example, n can range from 4 to 20. In some embodiments, n is chosen from 4, 8, 12, 16, and 20. For example, n can be 16.

The variable m is an integer from 1 to 3. In some embodiments, m is 1.

A blood-brain barrier agent, as used herein, is any polypeptide or non-polypeptide ligand that can cross the blood-brain barrier. In some embodiments, a blood-brain barrier agent has a cognate receptor on brain cells or can bind to such receptors. In some embodiments, the blood-brain barrier agent comprises a transferring-receptor binding site of a transferrin. In some embodiments, the blood-brain barrier agent comprises a receptor binding domain of an apolipoprotein. A receptor binding domain of an apolipoprotein, for example, can be chosen from the receptor binding domain of ApoA, ApoB, ApoC, ApoD, ApoE, ApoE2, ApoE3, ApoE4, and combinations thereof. In some embodiments, the receptor binding domain of an apoliprotein is chosen from the receptor binding domain of ApoB and ApoE.

In some embodiments, the blood-brain barrier agent comprises a sequence having at least 80% (e.g., at least 85%; at least 90%; at least 92%; at least 95%; at least 98%; and at least 99%) sequence identity to:

(SEQ ID NO: 13)
L-R-V-R-L-A-S-H-L-R-K-L-R-K-R-L-L-R-D-A;
(SEQ ID NO: 14)
S-S-V-I-D-A-L-Q-Y-K-L-E-G-T-T-R-L-T-R-K-R-G-L-K-L-
A-T-A-L-S-L-S-N-K-F-V-E-G-S-H;
(SEQ ID NO: 15)
Y-P-A-K-P-E-A-P-G-E-D-A-S-P-E-E-L-S-R-Y-Y-A-S-L-R-
H-Y-L-N-L-V-T-R-Q-R-Y*;
(SEQ ID NO: 16)
A-K-P-E-A-P-G-E-D-A-S-P-E-E-L-S-R-Y-Y-A-S-L-R-H-Y-
L-N-L-V-T-R-Q-R-Y*;
(SEQ ID NO: 17)
Y-P-S-D-P-D-N-P-G-E-D-A-P-A-E-D-L-A-R-Y-Y-S-A-L-R-
H-Y-I-N-L-I-T-R-Q-R-Y*;
or
(SEQ ID NO: 18)
A-P-L-E-P-V-Y-P-G-D-D-A-T-P-E-Q-M-A-Q-Y-A-A-E-L-R-
R-Y-I-N-M-L-T-R-P-R-Y*,
(SEQ ID NO: 19)
L-R-K-L-R-K-R-L-L-R-L-R-K-L-R-K-R-L-L-R

wherein Y* is tyrosine or a tyrosine derivative (e.g., an amidated tyrosine). See, e.g., Ballantyne, G. H., Obesity Surgery, 16:651-658 2006; and WO 2011/008823, both of which are incorporated by reference in their entirety.

In some embodiments, the blood-brain barrier agent includes a polypeptide comprising the following sequence:

(SEQ ID NO: 20)
L-R-X1-R-X2-X3-X4-H-L-R-X5-X6-X7-K-R-L-X8-R-D-X9

wherein:
X1 is selected from the group consisting of A, L, S, and V;
X2 is selected from the group consisting of L and M;
X3 is selected from the group consisting of A and S;
X4 is selected from the group consisting of N, S, and T;
X5 is selected from the group consisting of K and N;
X6 is selected from the group consisting of L, M, and V;
X7 is selected from the group consisting of R and P;
X8 is selected from the group consisting of L and M; and
X9 is selected from the group consisting of A and L.

Non-limiting examples of a blood-brain barrier agents according to this sequence include:

(SEQ ID NO: 13)
L-R-V-R-L-A-S-H-L-R-K-L-R-K-R-L-L-R-D-A;
(SEQ ID NO: 21)
L-R-S-R-L-A-S-H-L-R-K-L-R-K-R-L-L-R-D-A;
(SEQ ID NO: 22)
L-R-V-R-M-A-S-H-L-R-K-L-R-K-R-L-L-R-D-A;
(SEQ ID NO: 23)
L-R-V-R-L-A-T-H-L-R-K-L-R-K-R-L-L-R-D-A;
(SEQ ID NO: 24)
L-R-V-R-L-A-S-H-L-R-K-L-P-K-R-L-L-R-D-A;
(SEQ ID NO: 25)
L-R-V-R-L-A-S-H-L-R-K-L-R-K-R-L-M-R-D-A;
(SEQ ID NO: 26)
L-R-V-R-L-A-S-H-L-R-N-L-R-K-R-L-L-R-D-A;
(SEQ ID NO: 27)
L-R-V-R-L-A-S-H-L-R-K-V-R-K-R-L-L-R-D-A;
(SEQ ID NO: 28)
L-R-V-R-M-S-S-H-L-R-K-L-R-K-R-L-L-R-D-A;
(SEQ ID NO: 29)
L-R-V-R-L-A-S-H-L-R-N-V-R-K-R-L-L-R-D-A;
(SEQ ID NO: 30)
L-R-V-R-L-A-S-H-L-R-N-M-R-K-R-L-L-R-D-A;
(SEQ ID NO: 31)
L-R-A-R-M-A-S-H-L-R-K-L-R-K-R-L-L-R-D-A;
(SEQ ID NO: 32)
L-R-V-R-L-S-S-H-L-R-K-L-R-K-R-L-M-R-D-A;
(SEQ ID NO: 33)
L-R-S-R-L-A-S-H-L-R-K-L-R-K-R-L-M-R-D-A;
(SEQ ID NO: 34)
L-R-V-R-L-S-S-H-L-P-K-L-R-K-R-L-L-R-D-A;
(SEQ ID NO: 35)
L-R-V-R-L-A-S-H-L-R-K-M-R-K-R-L-M-R-D-A;
(SEQ ID NO: 36)
L-R-V-R-L-A-S-H-L-R-N-L-P-K-R-L-L-R-D-A;
(SEQ ID NO: 37)
L-R-L-R-L-A-S-H-L-R-K-L-R-K-R-L-L-R-D-L;
and
(SEQ ID NO: 38)
L-R-V-R-L-A-N-H-L-R-K-L-R-K-R-L-L-R-D-L.

In some embodiments, the blood-brain barrier agent is less than 100 amino acids in length (e.g., less than 90 amino acids in length; less than 80 amino acids in length; less than 75 amino acids in length; less than 70 amino acids in length; less than 65 amino acids in length; less than 62 amino acids in length; less than 60 amino acids in length; less than 55 amino acids in length; less than 50 amino acids in length; and less than 45 amino acids in length).

“Percent sequence identity” refers to the degree of sequence identity between any given reference sequence, e.g., SEQ ID NO:13, and a candidate blood-brain barrier agent sequence. A candidate sequence typically has a length that is from 80 percent to 200 percent of the length of the reference sequence (e.g., 82, 85, 87, 89, 90, 93, 95, 97, 99, 100, 105, 110, 115, 120, 130, 140, 150, 160, 170, 180, 190, or 200 percent of the length of the reference sequence). A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10; gap extension penalty: 5.0; and weight transitions: yes. For fast pairwise alignment of peptide sequences, the following parameters are used: word size: 1; window size: 5; scoring method: percentage; number of top diagonals: 5; gap penalty: 3. For multiple alignment of peptide sequences, the following parameters are used: weight matrix: blosum; gap opening penalty: 10; gap extension penalty: 0.5; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some embodiments, a peptide can be chosen from:

(SEQ ID NO: 39)
K-K-K-K-L-R-V-R-L-A-S-H-L-R-K-L-R-K-R-L-L-R-D-A;
(SEQ ID NO: 40)
K-K-K-K-K-K-K-K-L-R-V-R-L-A-S-H-L-R-K-L-R-K-R-L-L-
R-D-A;
(SEQ ID NO: 41)
K-K-K-K-K-K-K-K-K-K-K-K-L-R-V-R-L-A-S-H-L-R-K-L-R-
K-R-L-L-R-D-A;
(SEQ ID NO: 42)
K-K-K-K-K-K-K-K-K-K-K-K-K-K-K-K-L-R-V-R-L-A-S-H-L-
R-K-L-R-K-R-L-L-R-D-A;
(SEQ ID NO: 43)
K-K-K-K-K-K-K-K-K-K-K-K-K-K-K-K-K-K-K-K-L-R-V-R-L-
A-S-H-L-R-K-L-R-K-R-L-L-R-D-A;
(SEQ ID NO: 44)
K-K-K-K-S-S-V-I-D-A-L-Q-Y-K-L-E-G-T-T-R-L-T-R-K-R-
G-L-K-L-A-T-A-L-S-L-S-N-K-F-V-E-G-S-H;
(SEQ ID NO: 45)
K-K-K-K-K-K-K-K-S-S-V-I-D-A-L-Q-Y-K-L-E-G-T-T-R-L-
T-R-K-R-G-L-K-L-A-T-A-L-S-L-S-N-K-F-V-E-G-S-H;
(SEQ ID NO: 46)
K-K-K-K-K-K-K-K-K-K-K-K-S-S-V-I-D-A-L-Q-Y-K-L-E-G-
T-T-R-L-T-R-K-R-G-L-K-L-A-T-A-L-S-L-S-N-K-F-V-E-G-
S-H;
(SEQ ID NO: 47)
K-K-K-K-K-K-K-K-K-K-K-K-K-K-K-K-S-S-V-I-D-A-L-Q-Y-
K-L-E-G-T-T-R-L-T-R-K-R-G-L-K-L-A-T-A-L-S-L-S-N-K-
F-V-E-G-S-H;
and
(SEQ ID NO: 48)
K-K-K-K-K-K-K-K-K-K-K-K-K-K-K-K-K-K-K-K-S-S-V-I-D-
A-L-Q-Y-K-L-E-G-T-T-R-L-T-R-K-R-G-L-K-L-A-T-A-L-S-
L-S-N-K-F-V-E-G-S-H.

In some embodiments, a peptide can be chosen from:

(SEQ ID NO: 140)
[X]ₙ-L-R-X1-R-X2-X3-X4-H-L-R-X5-X6-X7-K-R-L-X8-R-
D-X9

wherein:
X is a hydrophilic amino acid;
n is an integer from 4 to 20;
X1 is selected from the group consisting of A, L, S, and V;
X2 is selected from the group consisting of L and M;
X3 is selected from the group consisting of A and S;
X4 is selected from the group consisting of N, S, and T;
X5 is selected from the group consisting of K and N;
X6 is selected from the group consisting of L, M, and V;
X7 is selected from the group consisting of R and P;
X8 is selected from the group consisting of L and M; and
X9 is selected from the group consisting of A and L.

Non-limiting examples of peptides according to this sequence include:

(SEQ ID NO: 39)
K-K-K-K-L-R-V-R-L-A-S-H-L-R-K-L-R-K-R-L-L-R-D-A;
(SEQ ID NO: 40)
K-K-K-K-K-K-K-K-L-R-V-R-L-A-S-H-L-R-K-L-R-K-R-L-L-
R-D-A;
(SEQ ID NO: 41)
K-K-K-K-K-K-K-K-K-K-K-K-L-R-V-R-L-A-S-H-L-R-K-L-R-
K-R-L-L-R-D-A;
(SEQ ID NO: 42)
K-K-K-K-K-K-K-K-K-K-K-K-K-K-K-K-L-R-V-R-L-A-S-H-L-
R-K-L-R-K-R-L-L-R-D-A;
(SEQ ID NO: 43)
K-K-K-K-K-K-K-K-K-K-K-K-K-K-K-K-K-K-K-K-L-R-V-R-L-
A-S-H-L-R-K-L-R-K-R-L-L-R-D-A;
(SEQ ID NO: 49)
K-K-K-K-L-R-S-R-L-A-S-H-L-R-K-L-R-K-R-L-L-R-D-A;
(SEQ ID NO: 50)
K-K-K-K-K-K-K-K-L-R-S-R-L-A-S-H-L-R-K-L-R-K-R-L-L-
R-D-A;
(SEQ ID NO: 51)
K-K-K-K-K-K-K-K-K-K-K-K-L-R-S-R-L-A-S-H-L-R-K-L-R-
K-R-L-L-R-D-A;
(SEQ ID NO: 52)
K-K-K-K-K-K-K-K-K-K-K-K-K-K-K-K-L-R-S-R-L-A-S-H-L-
R-K-L-R-K-R-L-L-R-D-A;
(SEQ ID NO: 53)
K-K-K-K-K-K-K-K-K-K-K-K-K-K-K-K-K-K-K-K-L-R-S-R-L-
A-S-H-L-R-K-L-R-K-R-L-L-R-D-A;
(SEQ ID NO: 54)
K-K-K-K-L-R-V-R-M-A-S-H-L-R-K-L-R-K-R-L-L-R-D-A;
(SEQ ID NO: 55)
K-K-K-K-K-K-K-K-L-R-V-R-M-A-S-H-L-R-K-L-R-K-R-L-L-
R-D-A;
(SEQ ID NO: 56)
K-K-K-K-K-K-K-K-K-K-K-K-L-R-V-R-M-A-S-H-L-R-K-L-R-
K-R-L-L-R-D-A;
(SEQ ID NO: 57)
K-K-K-K-K-K-K-K-K-K-K-K-K-K-K-K-L-R-V-R-M-A-S-H-L-
R-K-L-R-K-R-L-L-R-D-A;
(SEQ ID NO: 58)
K-K-K-K-K-K-K-K-K-K-K-K-K-K-K-K-K-K-K-K-L-R-V-R-M-
A-S-H-L-R-K-L-R-K-R-L-L-R-D-A;
(SEQ ID NO: 59)
K-K-K-K-L-R-V-R-L-A-T-H-L-R-K-L-R-K-R-L-L-R-D-A;
(SEQ ID NO: 60)
K-K-K-K-K-K-K-K-L-R-V-R-L-A-T-H-L-R-K-L-R-K-R-L-L-
R-D-A;
(SEQ ID NO: 61)
K-K-K-K-K-K-K-K-K-K-K-K-L-R-V-R-L-A-T-H-L-R-K-L-R-
K-R-L-L-R-D-A;
(SEQ ID NO: 62)
K-K-K-K-K-K-K-K-K-K-K-K-K-K-K-K-L-R-V-R-L-A-T-H-L-
R-K-L-R-K-R-L-L-R-D-A;
(SEQ ID NO: 63)
K-K-K-K-K-K-K-K-K-K-K-K-K-K-K-K-K-K-K-K-L-R-V-R-L-
A-T-H-L-R-K-L-R-K-R-L-L-R-D-A;
(SEQ ID NO: 64)
K-K-K-K-L-R-V-R-L-A-S-H-L-R-K-L-P-K-R-L-L-R-D-A;
(SEQ ID NO: 65)
K-K-K-K-K-K-K-K-L-R-V-R-L-A-S-H-L-R-K-L-P-K-R-L-L-
R-D-A;
(SEQ ID NO: 66)
K-K-K-K-K-K-K-K-K-K-K-K-L-R-V-R-L-A-S-H-L-R-K-L-P-
K-R-L-L-R-D-A;
(SEQ ID NO: 67)
K-K-K-K-K-K-K-K-K-K-K-K-K-K-K-K-L-R-V-R-L-A-S-H-L-
R-K-L-P-K-R-L-L-R-D-A;
(SEQ ID NO: 68)
K-K-K-K-K-K-K-K-K-K-K-K-K-K-K-K-K-K-K-K-L-R-V-R-L-
A-S-H-L-R-K-L-P-K-R-L-L-R-D-A;
(SEQ ID NO: 69)
K-K-K-K-L-R-V-R-L-A-S-H-L-R-K-L-R-K-R-L-M-R-D-A;
(SEQ ID NO: 70)
K-K-K-K-K-K-K-K-L-R-V-R-L-A-S-H-L-R-K-L-R-K-R-L-M-
R-D-A;
(SEQ ID NO: 71)
K-K-K-K-K-K-K-K-K-K-K-K-L-R-V-R-L-A-S-H-L-R-K-L-R-
K-R-L-M-R-D-A;
(SEQ ID NO: 72)
K-K-K-K-K-K-K-K-K-K-K-K-K-K-K-K-L-R-V-R-L-A-S-H-L-
R-K-L-R-K-R-L-M-R-D-A;
(SEQ ID NO: 73)
K-K-K-K-K-K-K-K-K-K-K-K-K-K-K-K-K-K-K-K-L-R-V-R-L-
A-S-H-L-R-K-L-R-K-R-L-M-R-D-A;
(SEQ ID NO: 74)
K-K-K-K-L-R-V-R-L-A-S-H-L-R-N-L-R-K-R-L-L-R-D-A;
(SEQ ID NO: 75)
K-K-K-K-K-K-K-K-L-R-V-R-L-A-S-H-L-R-N-L-R-K-R-L-L-
R-D-A;
(SEQ ID NO: 76)
K-K-K-K-K-K-K-K-K-K-K-K-L-R-V-R-L-A-S-H-L-R-N-L-R-
K-R-L-L-R-D-A;
(SEQ ID NO: 77)
K-K-K-K-K-K-K-K-K-K-K-K-K-K-K-K-L-R-V-R-L-A-S-H-L-
R-N-L-R-K-R-L-L-R-D-A;
(SEQ ID NO: 78)
K-K-K-K-K-K-K-K-K-K-K-K-K-K-K-K-K-K-K-K-L-R-V-R-L-
A-S-H-L-R-N-L-R-K-R-L-L-R-D-A;
(SEQ ID NO: 79)
K-K-K-K-L-R-V-R-L-A-S-H-L-R-K-V-R-K-R-L-L-R-D-A;
(SEQ ID NO: 80)
K-K-K-K-K-K-K-K-L-R-V-R-L-A-S-H-L-R-K-V-R-K-R-L-L-
R-D-A;
(SEQ ID NO: 81)
K-K-K-K-K-K-K-K-K-K-K-K-L-R-V-R-L-A-S-H-L-R-K-V-R-
K-R-L-L-R-D-A;
(SEQ ID NO: 82)
K-K-K-K-K-K-K-K-K-K-K-K-K-K-K-K-L-R-V-R-L-A-S-H-L-
R-K-V-R-K-R-L-L-R-D-A;
(SEQ ID NO: 83)
K-K-K-K-K-K-K-K-K-K-K-K-K-K-K-K-K-K-K-K-L-R-V-R-L-
A-S-H-L-R-K-V-R-K-R-L-L-R-D-A;
(SEQ ID NO: 84)
K-K-K-K-L-R-V-R-M-S-S-H-L-R-K-L-R-K-R-L-L-R-D-A;
(SEQ ID NO: 85)
K-K-K-K-K-K-K-K-L-R-V-R-M-S-S-H-L-R-K-L-R-K-R-L-L-
R-D-A;
(SEQ ID NO: 86)
K-K-K-K-K-K-K-K-K-K-K-K-L-R-V-R-M-S-S-H-L-R-K-L-R-
K-R-L-L-R-D-A;
(SEQ ID NO: 87)
K-K-K-K-K-K-K-K-K-K-K-K-K-K-K-K-L-R-V-R-M-S-S-H-L-
R-K-L-R-K-R-L-L-R-D-A;
(SEQ ID NO: 88)
K-K-K-K-K-K-K-K-K-K-K-K-K-K-K-K-K-K-K-K-L-R-V-R-M-
S-S-H-L-R-K-L-R-K-R-L-L-R-D-A;
(SEQ ID NO: 89)
K-K-K-K-L-R-V-R-L-A-S-H-L-R-N-V-R-K-R-L-L-R-D-A;
(SEQ ID NO: 90)
K-K-K-K-K-K-K-K-L-R-V-R-L-A-S-H-L-R-N-V-R-K-R-L-L-
R-D-A;
(SEQ ID NO: 91)
K-K-K-K-K-K-K-K-K-K-K-K-L-R-V-R-L-A-S-H-L-R-N-V-R-
K-R-L-L-R-D-A;
(SEQ ID NO: 92)
K-K-K-K-K-K-K-K-K-K-K-K-K-K-K-K-L-R-V-R-L-A-S-H-L-
R-N-V-R-K-R-L-L-R-D-A;
(SEQ ID NO: 93)
K-K-K-K-K-K-K-K-K-K-K-K-K-K-K-K-K-K-K-K-L-R-V-R-L-
A-S-H-L-R-N-V-R-K-R-L-L-R-D-A;
(SEQ ID NO: 94)
K-K-K-K-L-R-V-R-L-A-S-H-L-R-N-M-R-K-R-L-L-R-D-A;
(SEQ ID NO: 95)
K-K-K-K-K-K-K-K-L-R-V-R-L-A-S-H-L-R-N-M-R-K-R-L-L-
R-D-A;
(SEQ ID NO: 96)
K-K-K-K-K-K-K-K-K-K-K-K-L-R-V-R-L-A-S-H-L-R-N-M-R-
K-R-L-L-R-D-A;
(SEQ ID NO: 97)
K-K-K-K-K-K-K-K-K-K-K-K-K-K-K-K-L-R-V-R-L-A-S-H-L-
R-N-M-R-K-R-L-L-R-D-A;
(SEQ ID NO: 98)
K-K-K-K-K-K-K-K-K-K-K-K-K-K-K-K-K-K-K-K-L-R-V-R-L-
A-S-H-L-R-N-M-R-K-R-L-L-R-D-A;
(SEQ ID NO: 99)
K-K-K-K-L-R-A-R-M-A-S-H-L-R-K-L-R-K-R-L-L-R-D-A;
(SEQ ID NO: 100)
K-K-K-K-K-K-K-K-L-R-A-R-M-A-S-H-L-R-K-L-R-K-R-L-L-
R-D-A;
(SEQ ID NO: 101)
K-K-K-K-K-K-K-K-K-K-K-K-L-R-A-R-M-A-S-H-L-R-K-L-R-
K-R-L-L-R-D-A;
(SEQ ID NO: 102)
K-K-K-K-K-K-K-K-K-K-K-K-K-K-K-K-L-R-A-R-M-A-S-H-L-
R-K-L-R-K-R-L-L-R-D-A;
(SEQ ID NO: 103)
K-K-K-K-K-K-K-K-K-K-K-K-K-K-K-K-K-K-K-K-L-R-A-R-M-
A-S-H-L-R-K-L-R-K-R-L-L-R-D-A;
(SEQ ID NO: 104)
K-K-K-K-L-R-V-R-L-S-S-H-L-R-K-L-R-K-R-L-M-R-D-A;
(SEQ ID NO: 105)
K-K-K-K-K-K-K-K-L-R-V-R-L-S-S-H-L-R-K-L-R-K-R-L-M-
R-D-A;
(SEQ ID NO: 106)
K-K-K-K-K-K-K-K-K-K-K-K-L-R-V-R-L-S-S-H-L-R-K-L-R-
K-R-L-M-R-D-A;
(SEQ ID NO: 107)
K-K-K-K-K-K-K-K-K-K-K-K-K-K-K-K-L-R-V-R-L-S-S-H-L-
R-K-L-R-K-R-L-M-R-D-A;
(SEQ ID NO: 108)
K-K-K-K-K-K-K-K-K-K-K-K-K-K-K-K-K-K-K-K-L-R-V-R-L-
S-S-H-L-R-K-L-R-K-R-L-M-R-D-A;
(SEQ ID NO: 109)
K-K-K-K-L-R-S-R-L-A-S-H-L-R-K-L-R-K-R-L-M-R-D-A;
(SEQ ID NO: 110)
K-K-K-K-K-K-K-K-L-R-S-R-L-A-S-H-L-R-K-L-R-K-R-L-M-
R-D-A;
(SEQ ID NO: 111)
K-K-K-K-K-K-K-K-K-K-K-K-L-R-S-R-L-A-S-H-L-R-K-L-R-
K-R-L-M-R-D-A;
(SEQ ID NO: 112)
K-K-K-K-K-K-K-K-K-K-K-K-K-K-K-K-L-R-S-R-L-A-S-H-L-
R-K-L-R-K-R-L-M-R-D-A;
(SEQ ID NO: 113)
K-K-K-K-K-K-K-K-K-K-K-K-K-K-K-K-K-K-K-K-L-R-S-R-L-
A-S-H-L-R-K-L-R-K-R-L-M-R-D-A;
(SEQ ID NO: 114)
K-K-K-K-L-R-V-R-L-S-S-H-L-P-K-L-R-K-R-L-L-R-D-A;
(SEQ ID NO: 115)
K-K-K-K-K-K-K-K-L-R-V-R-L-S-S-H-L-P-K-L-R-K-R-L-L-
R-D-A;
(SEQ ID NO: 116)
K-K-K-K-K-K-K-K-K-K-K-K-L-R-V-R-L-S-S-H-L-P-K-L-R-
K-R-L-L-R-D-A;
(SEQ ID NO: 117)
K-K-K-K-K-K-K-K-K-K-K-K-K-K-K-K-L-R-V-R-L-S-S-H-L-
P-K-L-R-K-R-L-L-R-D-A;
(SEQ ID NO: 118)
K-K-K-K-K-K-K-K-K-K-K-K-K-K-K-K-K-K-K-K-L-R-V-R-L-
S-S-H-L-P-K-L-R-K-R-L-L-R-D-A;
(SEQ ID NO: 119)
K-K-K-K-L-R-V-R-L-A-S-H-L-R-K-M-R-K-R-L-M-R-D-A;
(SEQ ID NO: 120)
K-K-K-K-K-K-K-K-L-R-V-R-L-A-S-H-L-R-K-M-R-K-R-L-M-
R-D-A;
(SEQ ID NO: 121)
K-K-K-K-K-K-K-K-K-K-K-K-L-R-V-R-L-A-S-H-L-R-K-M-R-
K-R-L-M-R-D-A;
(SEQ ID NO: 122)
K-K-K-K-K-K-K-K-K-K-K-K-K-K-K-K-L-R-V-R-L-A-S-H-L-
R-K-M-R-K-R-L-M-R-D-A;
(SEQ ID NO: 123)
K-K-K-K-K-K-K-K-K-K-K-K-K-K-K-K-K-K-K-K-L-R-V-R-L-
A-S-H-L-R-K-M-R-K-R-L-M-R-D-A;
(SEQ ID NO: 124)
K-K-K-K-L-R-V-R-L-A-S-H-L-R-N-L-P-K-R-L-L-R-D-A;
(SEQ ID NO: 125)
K-K-K-K-K-K-K-K-L-R-V-R-L-A-S-H-L-R-N-L-P-K-R-L-L-
R-D-A;
(SEQ ID NO: 126)
K-K-K-K-K-K-K-K-K-K-K-K-L-R-V-R-L-A-S-H-L-R-N-L-P-
K-R-L-L-R-D-A;
(SEQ ID NO: 127)
K-K-K-K-K-K-K-K-K-K-K-K-K-K-K-K-L-R-V-R-L-A-S-H-L-
R-N-L-P-K-R-L-L-R-D-A;
(SEQ ID NO: 128)
K-K-K-K-K-K-K-K-K-K-K-K-K-K-K-K-K-K-K-K-L-R-V-R-L-
A-S-H-L-R-N-L-P-K-R-L-L-R-D-A;
(SEQ ID NO: 129)
K-K-K-K-L-R-L-R-L-A-S-H-L-R-K-L-R-K-R-L-L-R-D-L;
(SEQ ID NO: 130)
K-K-K-K-K-K-K-K-L-R-L-R-L-A-S-H-L-R-K-L-R-K-R-L-L-
R-D-L;
(SEQ ID NO: 131)
K-K-K-K-K-K-K-K-K-K-K-K-L-R-L-R-L-A-S-H-L-R-K-L-R-
K-R-L-L-R-D-L;
(SEQ ID NO: 132)
K-K-K-K-K-K-K-K-K-K-K-K-K-K-K-K-L-R-L-R-L-A-S-H-L-
R-K-L-R-K-R-L-L-R-D-L;
(SEQ ID NO: 133)
K-K-K-K-K-K-K-K-K-K-K-K-K-K-K-K-K-K-K-K-L-R-L-R-L-
A-S-H-L-R-K-L-R-K-R-L-L-R-D-L;
(SEQ ID NO: 134)
K-K-K-K-L-R-V-R-L-A-N-H-L-R-K-L-R-K-R-L-L-R-D-L;
(SEQ ID NO: 135)
K-K-K-K-K-K-K-K-L-R-V-R-L-A-N-H-L-R-K-L-R-K-R-L-L-
R-D-L;
(SEQ ID NO: 136)
K-K-K-K-K-K-K-K-K-K-K-K-L-R-V-R-L-A-N-H-L-R-K-L-R-
K-R-L-L-R-D-L;
(SEQ ID NO: 137)
K-K-K-K-K-K-K-K-K-K-K-K-K-K-K-K-L-R-V-R-L-A-N-H-L-
R-K-L-R-K-R-L-L-R-D-L;
(SEQ ID NO: 138)
K-K-K-K-K-K-K-K-K-K-K-K-K-K-K-K-K-K-K-K-L-R-V-R-L-
A-N-H-L-R-K-L-R-K-R-L-L-R-D-L.

### Active Agents

The methods provided herein provide transport of an active agent across the BBB. In some embodiments, an active agent is a biologically active molecule or an imaging agent.

As used herein, a “biologically active molecule” includes any molecule which, if transported across the blood-brain barrier, can have a biological effect. Examples of biologically active molecules include polypeptides, which include functional domains of biologically active molecules, particular examples include growth factors, enzymes, transcription factors, toxins, antigenic peptides (as for vaccines), antibodies, and antibody fragments. For example, brain derived neurotrophic factor, fibroblast growth factor (e.g., (FGF)-2 or multiple FGFs), nerve growth factor, neurotrophin (e.g., NT-3 and NT-4/5), glial derived neurotrophic factor, ciliary neurotrophic factor, neurturin, neuregulins, interleukins, transforming growth factor (e.g., TGF-α and TGF-β), vasoactibe intestinal peptide, epidermal growth factor (EGF), erythropoietin, heptocytel growth factor, platelet derived growth factor, artemin, persephin, netrins, cardiotrophin-1, stem cell factor, midkine, pleiotrophin, bone morphogenic proteins, saposins, semaporins, leukemia inhibitory factor, anti-Aβ, anti-HER2, anti-EGF, anti-nogo A, anti-TRAIL (tumor necrosis factor-related apoptosis-inducing ligand), anti-α-synuclein, anti-htt, anti-prion, anti-West Nile virus, αL-iduronidase, iduronate-2-sulfatase, N-acetyl-galactosamine-6-sulfatase, arylsulfatase B, acid α-glucosidase, and acid sphingomyelinase (See, Pardridge, W. M., Bioconjug. Chem. 19(7): 1327-38 2008). Additional examples of biologically active molecules include oligonucleotides, such as natural or engineered plasmids, coding DNA sequences, antisense DNA sequences, mRNAs, antisense RNA sequences, RNAis, and siRNAs; carbohydrates; lipids; and glycolipids.

Further examples of biologically active molecules include small molecules, including therapeutic agents, in particular those with low blood-brain barrier permeability. Some examples of these therapeutic agents include cancer drugs, such as daunorubicin, doxorubicin, and toxic chemicals which, because of the lower dosage that can be administered by this method, can now be more safely administered. For example, a therapeutic agent can include bevacizumab, irinotecan, zoledronate, temozolomide, taxol, methotrexate, and cisplatin.

In another embodiment, the therapeutic agent can include a broad-spectrum antibiotic (e.g., cefotaxime, ceftriaxone, ampicillin and vancomycin); an antiviral agent (e.g., acyclovir); acetazolamide; carbamazepine; clonazepam; clorazepate dipotassium; diazepam; divalproex sodium; ethosuximide; felbamate; fosphenytoin sodium; gabapentin; lamotrigine; levetiracetam; lorazepam; oxcarbazepine; phenobarbital; phenytoin; phenytoin sodium; pregabalin; primidone; tiagabine hydrochloride; topiramate; trimethadione; valproic acid; zonisamide; copaxone; tysabri; novantrone; donezepil HCL; rivastigmine; galantamine; memantine; levodopa; carbidopa; parlodel, permax, requip, mirapex; Symmetrel; artane; cogentin; eldepryl; and deprenyl.

Numerous other examples of biologically active molecules will be apparent to the skilled artisan.

Yet another example of a biologically active molecule is an antigenic peptide. Antigenic peptides can be administered to provide immunological protection when imported by cells involved in the immune response. Other examples include immunosuppressive peptides (e.g., peptides that block autoreactive T cells, which peptides are known in the art).

Polypeptides from a few amino acids to about a thousand amino acids can be used. In some embodiments, the size range for polypeptides is from a few amino acids to about 250 amino acids (e.g., about 3 to about 250 amino acids; about 20 to about 250 amino acids; about 50 to about 250 amino acids; about 100 to about 250 amino acids; about 150 to about 250 amino acids; about 3 amino acids to about 200 amino acids; about 3 amino acids to about 150 amino acids; about 3 amino acids to about 175 amino acids; about 3 amino acids to about 125 amino acids; about 25 amino acids to about 200 amino acids; about 50 amino acids to about 150 amino acids; and about 75 amino acids to about 225 amino acids). For any molecule, size ranges can be up to about a molecular weight of about 1 million. In some embodiments, the size ranges up to a molecular weight of about 25,000, and in particular embodiments, the size ranges can be up to a molecular weight of about 3,000.

By “antisense” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004; Agrawal et al., U.S. Pat. No. 5,591,721; Agrawal, U.S. Pat. No. 5,652,356). Typically, antisense molecules will be complementary to a target sequence along a single contiguous sequence of the antisense molecule. However, in certain embodiments, an antisense molecule may bind to a substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

RNA interference (RNAi) and short intervening RNA (siRNA) sequences can be used to modulate (e.g., inhibit) gene expression (see, e.g., Elbashir et al., 2001, Nature, 411, 494 498; and Bass, 2001, Nature, 411, 428 429; Bass, 2001, Nature, 411, 428 429; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914). In one embodiment, a siRNA molecule comprises a double stranded RNA wherein one strand of the RNA is complimentary to the RNA of interest. In another embodiment, a siRNA molecule comprises a double stranded RNA wherein one strand of the RNA comprises a portion of a sequence of an RNA of interest. In yet another embodiment, a siRNA molecule of the disclosure comprises a double stranded RNA wherein both strands of RNA are connected by a non-nucleotide linker Alternately, a siRNA molecule of the disclosure comprises a double stranded RNA wherein both strands of RNA are connected by a nucleotide linker, such as a loop or stem loop structure.

The term “antibody” as used herein refers to immunoglobulin molecules and immunologically active portions of immunoglobulin molecules (i.e., molecules that contain an antigen binding site that specifically binds to a peptide). An antibody can be a monoclonal antibody, a polyclonal antibody, a humanized antibody, a fully human antibody, a single chain antibody, a chimeric antibody, or a fragment thereof. The term “antibody fragment” of a full length antibody refers to one or more fragments of a full-length antibody that retain the ability to specifically bind to a target of interest.

An imaging agent, as used herein, can be any chemical or substance which is used to provide a signal or contrast in imaging. The signal enhancing domain can be an organic molecule, metal ion, salt or chelate, particle (particularly iron particle), or labeled peptide, protein, polymer or liposome. For example, an imaging agent can include one or more of a radionuclide, a paramagnetic metal, a fluorochrome, a dye, and an enzyme substrate.

In some embodiments, the imaging agent is a physiologically compatible metal chelate compound consisting of one or more cyclic or acyclic organic chelating agents complexed to one or more metal ions with atomic numbers 21-29, 42, 44, or 57-83.

For x-ray imaging, the imaging agent may consist of iodinated organic molecules or chelates of heavy metal ions of atomic numbers 57 to 83. In some embodiments, the imaging agent is ¹²⁵I-IgG. Examples of suitable compounds are described in M. Sovak, ed., “Radiocontrast Agents,” Springer-Verlag, pp. 23-125 (1984) and U.S. Pat. No. 4,647,447.

For ultrasound imaging, the imaging agent can consist of gas-filled bubbles such as Albunex, Echovist, or Levovist, or particles or metal chelates where the metal ions have atomic numbers 21-29, 42, 44 or 57-83. Examples of suitable compounds are described in Tyler et al., Ultrasonic Imaging, 3, pp. 323-29 (1981) and D. P. Swanson, “Enhancement Agents for Ultrasound: Fundamentals,” Pharmaceuticals in Medical Imaging, pp. 682-87. (1990).

For nuclear radiopharmaceutical imaging or radiotherapy, the imaging agent can consist of a radioactive molecule. In some embodiments, the chelates of Tc, Re, Co, Cu, Au, Ag, Pb, Bi, In, and Ga can be used. In some embodiments, the chelates of Tc-99m can be used. Examples of suitable compounds are described in Rayudu GVS, Radiotracers for Medical Applications, I, pp. 201 and D. P. Swanson et al., ed., Pharmaceuticals in Medical Imaging, pp. 279-644 (1990).

For ultraviolet/visible/infrared light imaging, the imaging agent can consist of any organic or inorganic dye or any metal chelate.

For MRI, the imaging agent can consist of a metal-ligand complex of a paramagnetic form of a metal ion with atomic numbers 21-29, 42, 44, or 57-83. In some embodiments, the paramagnetic metal is chosen from: Gd(III), Fe(III), Mn(II and III), Cr(III), Cu(II), Dy(III), Tb(III), Ho(III), Er(III) and Eu(III). Many suitable chelating ligands for MRI agents are known in the art. These can also be used for metal chelates for other forms of biological imaging. For example, an imaging agent can include: Gadovist, Magnevist, Dotarem, Omniscan, and ProHance.

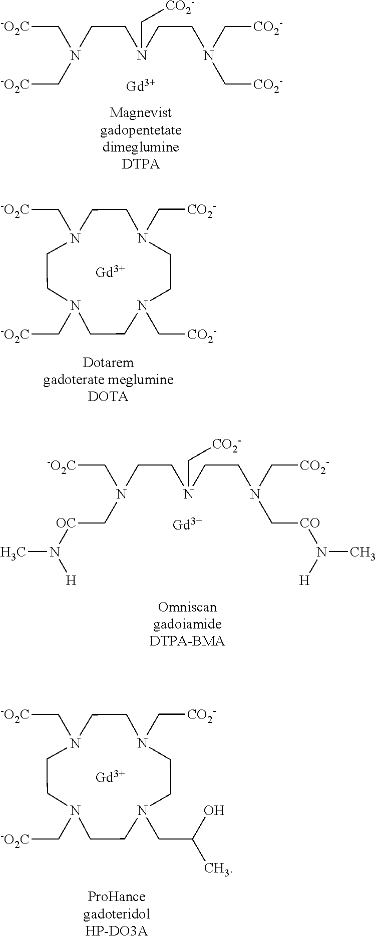

### Methods of Use

Provided herein are methods of transporting an active agent across the BBB of a patient. In some embodiments, the method comprises administering a peptide as provided herein to the patient followed by administration of an active agent as provided herein.

An active agent can be administered to a patient from about 5 minutes to about 6 hours after administration of the peptide (e.g., about 5 minutes to about 5.5 hours; about 5 minutes to about 5 hours; about 5 minutes to about 4.5 hours; about 5 minutes to about 4 hours; about 5 minutes to about 3.5 hours; about 5 minutes to about 3 hours; about 5 minutes to about 2 hours; about 5 minutes to about 1.5 hours; about 5 minutes to about 1 hour; about 5 minutes to about 45 minutes; about 5 minutes to about 35 minutes; about 5 minutes to about 30 minutes; about 5 minutes to about 25 minutes; about 5 minutes to about 20 minutes; about 5 minutes to about 15 minutes; about 10 minutes to about 6 hours; about 15 minutes to about 6 hours; about 30 minutes to about 6 hours; about 45 minutes to about 6 hours; about 1 hour to about 6 hours; about 1.5 hours to about 6 hours; about 2 hours to about 6 hours; about 3 hours to about 6 hours; about 10 minutes to about 1 hour; about 15 minutes to about 45 minutes; about 20 minutes to about 50 minutes; about 30 minutes to about 1.5 hours; about 25 minutes to about 55 minutes; and about 10 minutes to about 30 minutes). For example, an active agent can be administered to a patient from about 5 minutes to about 2 hours after administration of the peptide. In some embodiments, an active agent is administered to a patient from about 10 minutes to about 1 hour after administration of the peptide.

In some embodiments, a synthetic peptide as provided herein can be administered with a natural polypeptide or antibody capable of binding to a protein at the blood-brain barrier. For example, the peptide can be administered with IgG, YY (PYY), neuropeptide Y (NPY), corticotropin releasing factor (CRF), and urocortin. In some embodiments, the peptide is administered with IgG.

A patient can include both mammals and non-mammals. Mammals include, for example, humans; nonhuman primates, e.g. apes and monkeys; cattle; horses; sheep; rats; mice; pigs; and goats. Non-mammals include, for example, fish and birds.

Transporting an active agent can include importing the molecule across the blood-brain barrier.

Further provided herein is a method of treating a brain disorder in a patient. The method can include administering to the patient a peptide as provided herein to the patient followed by administration of an biologically active molecule as provided herein. In some embodiments, the biologically active molecule is a therapeutic agent. In some embodiments, the peptide is administered in combination with a natural polypeptide or antibody. For example, the peptide can be administered with a natural polypeptide, an antibody, or the patient's own serum (e.g., containing IgG). In some embodiments, the natural polypeptide, antibody, or patient's serum can also function as a therapeutic agent.

In some embodiments, the brain disorder is selected from: meningitis, epilepsy, multiple sclerosis, neuromyelitis optica, late-stage neurological trypanosomiasis, Parkinson's, progressive multifocal leukoencephalopathy, De Vivo disease, Alzheimer's disease, HIV Encephalitis, and cancer. For example, a brain disorder can be a brain cancer, for example astrocytoma, medulloblastoma, glioma, ependymoma, germinoma (pinealoma), glioblastoma multiform, oligodendroglioma, schwannoma, retinoblastoma, and congenital tumors; or a cancer of the spinal cord, e.g., neurofibroma, meningioma, glioma, and sarcoma.

This disclosure also provides a method of imaging the central nervous system of a patient. In some embodiments, the method can include administering to the patient a peptide as provided herein to the patient followed by administration of an imaging agent as provided herein, and imaging the central nervous system of the patient. In some embodiments, the peptide is administered in combination with a natural polypeptide and/or antibody.

A peptide and an active agent can be administered by any route, e.g., IV, intramuscular, SC, oral, intranasal, inhalation, transdermal, and parenteral. In some embodiments, a peptide and active agent are administered by IV.

A peptide and/or an active agent can be formulated with a pharmaceutically acceptable carrier selected on the basis of the selected route of administration and standard pharmaceutical practice. A peptide and/or an active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, a peptide and/or an active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of a peptide and/or an active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, a peptide and/or an active agent may be combined with one or more solid inactive ingredients for the preparation of tablets, capsules, pills, powders, granules or other suitable oral dosage forms. For example, a peptide and/or an active agent may be combined with at least one excipient such as fillers, binders, humectants, disintegrating agents, solution retarders, absorption accelerators, wetting agents absorbents or lubricating agents.

The specific dose of a peptide and/or an active agent will, of course, be determined by the particular circumstances of the individual patient including the size, weight, age and sex of the patient, the nature and stage of the disease being treated, the aggressiveness of the disease disorder, and the route of administration of the compound.

An “effective amount” of an active agent (e.g., an imaging agent) provided herein is typically one which is sufficient to the desired effect of the agent (e.g., detection of an imaging agent) and may vary according to the detection method utilized and the detection limit of the agent.

A “therapeutically effective” amount of an agent (e.g., a biologically active molecule) provided herein is typically one which is sufficient to achieve the desired effect and may vary according to the nature and severity of the disease condition, and the potency of the agent. It will be appreciated that different concentrations may be employed for prophylaxis than for treatment of an active disease.

### Pharmaceutical Compositions

Provided herein are pharmaceutical compositions comprising a peptide or an active agent.

The pharmaceutical compositions provided herein contain a peptide or an agent in an amount that results in transportation of the agent across the blood-brain barrier, and a pharmaceutically acceptable carrier. Pharmaceutical carriers suitable for administration of a peptide or an active agent provided herein include any such carriers known to those skilled in the art to be suitable for the particular mode of administration.

The compositions can be, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration and intraperitoneal injection, as well as transdermal patch preparation, dry powder inhalers, and ointments (see, e.g., Ansel Introduction to Pharmaceutical Dosage Forms, Fourth Edition 1985, 126).

The concentration of an active agent administered to the patient will depend on absorption, inactivation and excretion rates of the compounds, the physicochemical characteristics of the compounds, the dosage schedule, and amount administered as well as other factors known to those of skill in the art.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. A peptide and/or an active agent is (are), in one embodiment, formulated and administered in unit-dosage forms or multiple-dosage forms. Unit-dose forms as used herein refers to physically discrete units suitable for human and animal patients and packaged individually as is known in the art. Each unit-dose contains a predetermined quantity of the peptide or active agent sufficient to produce the desired effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing a peptide or an active agent in the range of 0.005% to 100% with the balance made up with a non-toxic carrier may be prepared. Methods for preparation of these compositions are known to those skilled in the art. The contemplated compositions may contain 0.001%-100% a peptide or an active agent, in one embodiment 0.1-95%, in another embodiment 75-85%.

### Kits

Also provided herein are kits. Typically, a kit includes a peptide, as provided herein and one or more active agents as provided herein. In some embodiments, a kit includes a peptide and a biologically active molecule and/or imaging agent. In certain embodiments, a kit can include one or more delivery systems, e.g., for a biologically active molecule, imaging agent, peptide, or any combination thereof, and directions for use of the kit (e.g., instructions for administering to a patient). In some embodiments, a kit can include a syringe comprising a peptide and a syringe comprising an active agent as provided herein. In some embodiments, the kit can include a peptide, one or more active agents and a label that indicates that the contents are to be administered to a patient prior to administration of an active agent (e.g., a biologically active molecule or imaging agent).

### EXAMPLES

### Example 1

### Transportation of a Dye Across the BBB

To explore whether K16ApoE (SEQ. ID. NO: 42) compromises integrity of the BBB, the peptide was first injected by iv followed by injection of Evan's Blue (EB; a routinely used small molecule blue dye with a molecular weight of 960.81) by iv into the femoral vein of a mouse. Cardiac perfusion was performed and the brains of the mice were removed and evaluated approximately 2 hours following injection of the dye. As controls, a peptide consisting of 16 lysine residues (K16) and a 20-amino acid peptide comprising the LDLR-binding domain of ApoE (ApoE-FITC; (SEQ ID NO:13)), were used. The particulars of each administration are as follows:
1) 67.5 nanomoles of K16 injected first, then ˜40 μL 2% EB was injected.
2) Same as 1 but K16 and EB mixed prior to injection.
3) 67.5 nanomoles of ApoE-FITC injected first, then ˜40 μL 2% EB was injected.
4) Same as 3 but ApoE-FITC peptide and EB was mixed prior to injection.
5) 67.5 nanomoles of K16ApoE injected first, then ˜40 μL 2% EB was injected.
6) Same as 5 but K16ApoE peptide and EB was mixed first before injection.

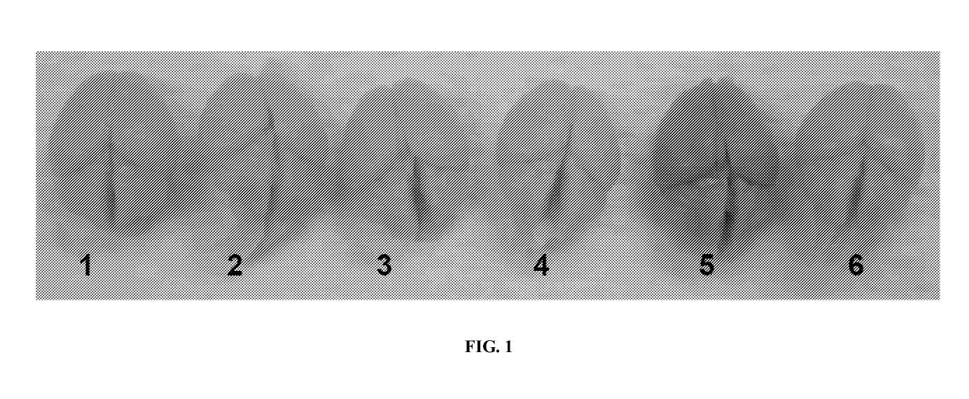

As is shown in FIG. 1, the EB dye crosses the BBB and is transported to the brain when K16ApoE is injected first followed by injection of the dye. This is evidenced by only brain number 5 showing the blue coloring of the EB dye.

### Example 2

### Transportation Across the BBB as a Function of the Amount of Peptide Administered

Using the method described in Example 1, various amounts of K16ApoE (SEQ. ID. NO: 42) was injected followed by administration of ˜40 μL 2% EB. The particulars of each administration are as follows:
1) EB alone.
2) 50 μg (11.25 nanomoles) K16ApoE first, followed by administration of EB.
3) 100 μg (22.5 nanomoles) K16ApoE first, followed by administration of EB.
4) 200 μg (45 nanomoles) K16ApoE first, followed by administration of EB.
5) 300 μg (67.5 nanomoles) K16ApoE first, followed by administration of EB.

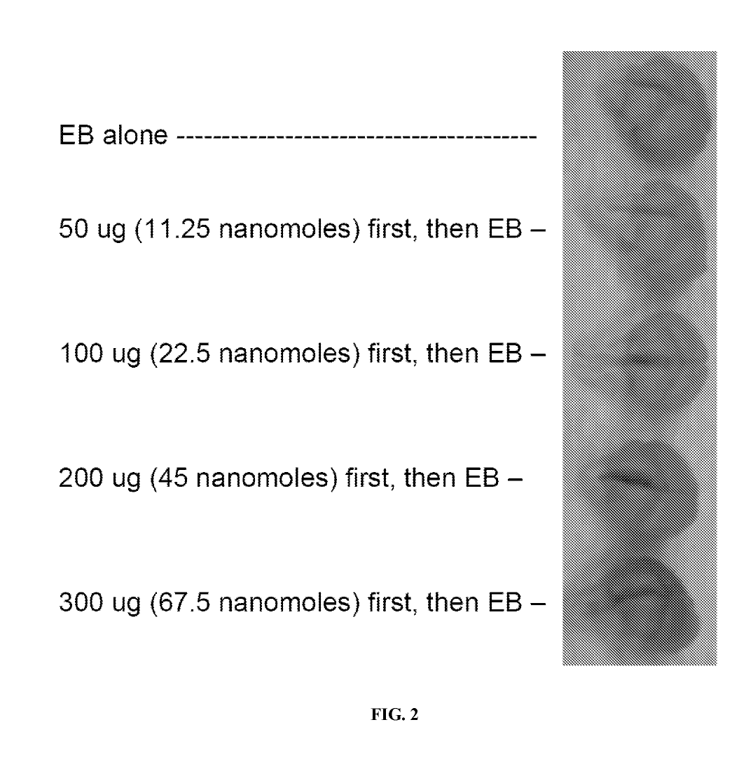

As shown in FIG. 2, no dye is observed in the brains prior to administration of 100 μg of K16ApoE. Each increasing amount of K16ApoE shows an increasing amount of EB dye present in the brain and therefore more intense blue coloring. Accordingly, these results indicate that increased brain uptake of EB is achieved with each higher dose of K16ApoE injected.

### Example 3

### Transportation of Compounds of Various Sizes Across the BBB

To evaluate delivery of molecules of various molecular weights across the blood brain barrier, the method of Example 1 was used with Evan's Blue (EB), MW 960.81 gmol; Crocein Scarlet, MW 584.54 g/mol; and Light green SF, MW 792.86 g/mol. In addition to evaluating the ability of K16ApoE to transport the molecules across the BBB, a combination of K16ApoE and IgG was also administered. The particulars of each administration are as follows:
1) Dye only (˜40 μL 2%).
2) Injection of K16ApoE (300 μg) first, followed by injection of dye (˜40 μL 2%).
3) Injection of (300 μg K16ApoE+300 μg IgG) first, followed by injection of dye (˜40 μL 2%)
4) Injection of 300 μg K16ApoE mixed with dye (˜40 μL 2%) prior to injection.

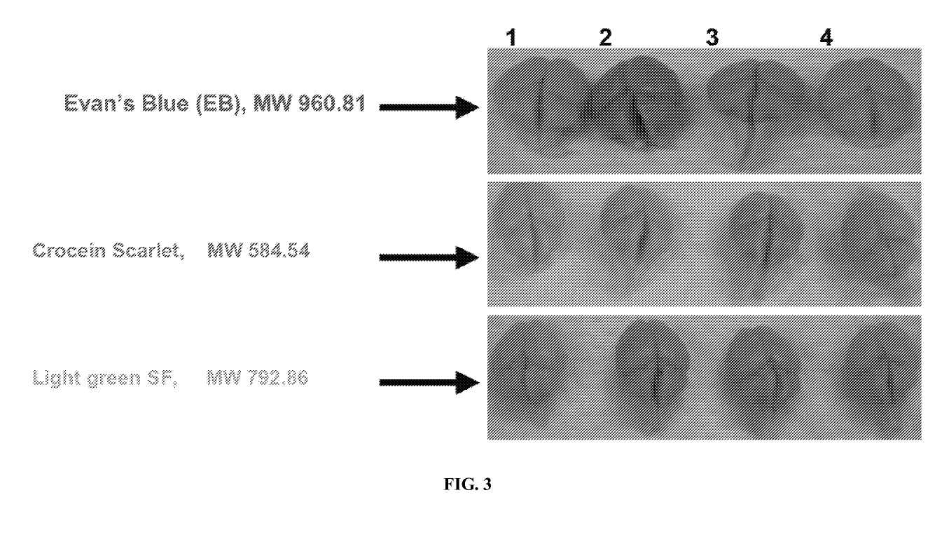

As show in FIG. 3, evidence of the dye in the brain is only observed in samples 2 and 3, where the peptide was injected prior to injection of dye. Accordingly, prior injection of K16ApoE or a mixture of K16ApoE+IgG allowed transport of molecules of various sizes to the brain. Interestingly, if the molecule to be delivered is mixed with the peptide and then injected, the molecule does not appear to cross the BBB.

### Example 4

### Evaluation of the Length of Time of BBB Opening Following Administration of Peptide

A series of mice were administered 300 μg of K16ApoE followed by administration of ˜40 μL 2% EB at various time points. As a control, only EB was injected, cardiac perfusion was done 6 hours after EB injection, followed by collection of brain. For the other images, 300 μg of K16ApoE was first injected, then EB was injected after 5 min, 10 min, 30 min, 1 h, 2 h and 4 h, respectively. Then, perfusion and collection of brain was done after 5 h 55 min, 5 h 50 min, 5 h 30 min, 5 h, 4 h and 2 h, respectively).

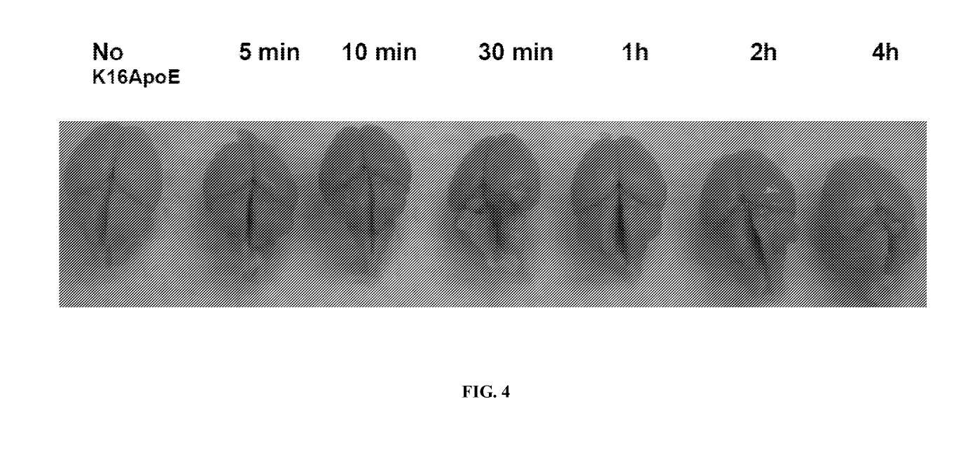

As shown in FIG. 4, no dye was observed in the control image. EB was, however, observed the 5 min, 10 min, 30 min samples, with decreased intensity of dye observed in the 1 h and 2 h samples. The 4 h samples showed little to no EB dye remaining. These results indicate that the BBB remains open for 30 min-1 h after injection of the peptide.

### Example 5

### Evaluation of Residence Time of Dye Following Transportation by the Peptide

A series of mice were administered 300 μg of K16ApoE followed by administration of ˜40 μL 2% EB 10 min after administration of the peptide. As a control, only EB was injected, cardiac perfusion was done 6 hours after EB injection, followed by collection of brain. For all other mice, perfusion and collection of brains was performed 15 min, 30 min, 1 h, 2 h, 4 h, 6 h, 12, h, and 24 h following administration of EB.

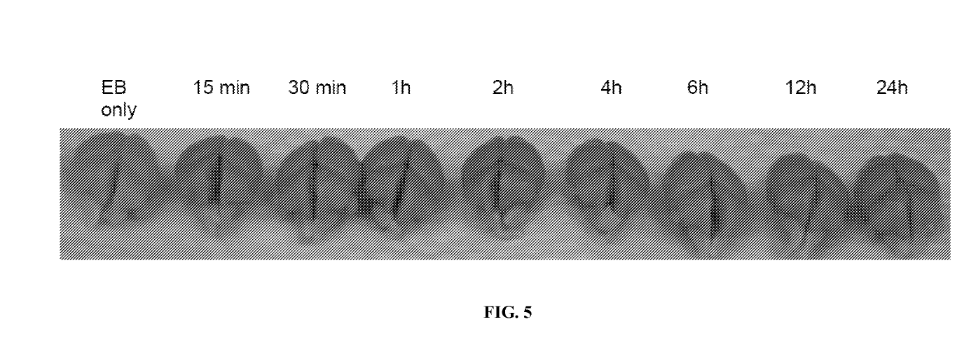

As shown in FIG. 5, no dye is observed in the control brain. Dye is observed, however, starting at 15 minutes post EB administration and remains intense up to 4 h after administration. Dye is still observed, although less intensely, up to 24 hours after administration of the dye. Accordingly, it appears that Evan's Blue (EB) delivered via K16ApoE is retained well in the brain for up to 4 h, and probably up to 24 h (longest time point tested) following administration of the dye.

### Example 6

### Comparison of Delivery of EB by K16ApoE and K26ApoE

This experiment was carried out to evaluate if lengthening the lysine-chain will have any effect on the delivery of small molecules to the brain.

˜40 μL of 2% EB was injected 10 min after injection of various amounts of K16ApoE (75 μg, 100 μg, 150 μg, 250 μg, and 300 μg) or K26ApoE (25 μg, 50 μg, 75 μg, 100 μg, 125 μg, and 150 μg). K26ApoE (SEQ ID NO: 139) having the sequence:

(SEQ ID NO: 139)
K-K-K-K-K-K-K-K-K-K-K-K-K-K-K-K-K-K-K-K-K-K-K-K-K-
K-L-R-V-R-L-A-S-H-L-R-K-L-R-K-R-L-L-R-D-A.

Brain tissue as was collected 1 h following EB injection. All injections were at the femoral vein.

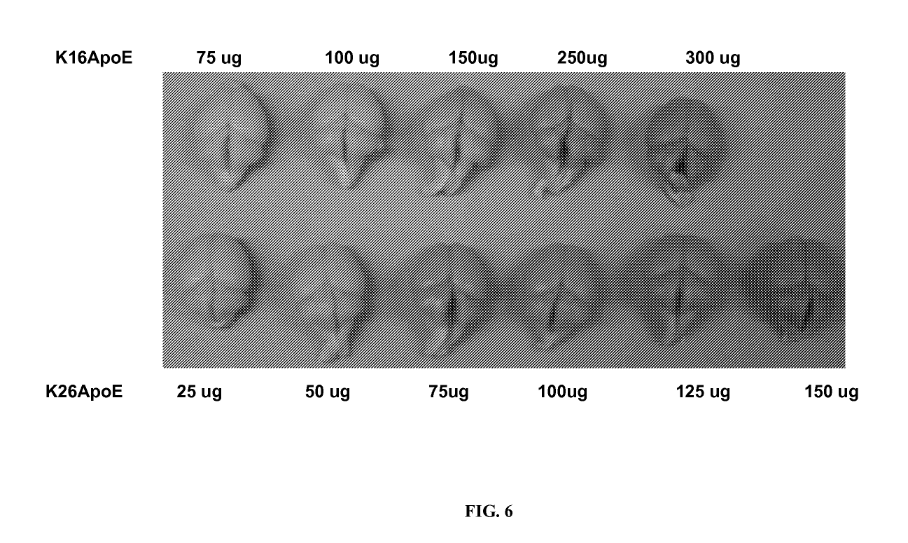

As shown in FIG. 6, the amount of dye transported across the BBB increases in with increasing amounts of peptide administered. In the case of K16ApoE, no dye was observed with 75 μg of peptide with only a small amount of dye present with 100 μg. Increasing EB is observed starting with 150 μg of peptide. In the case of K26ApoE, no dye was observed with 25 μg of peptide with only a small amount of dye present with 50 μg. Increasing EB is observed starting with 75 μg of peptide. These results suggest K26ApoE is approximately two-fold more effective than K16ApoE in delivering EB to the brain.

### Example 7

### Tissue Distribution of Evans Blue (EB) after Delivery Via K16ApoE

Mice were injected with various amounts of K16ApoE peptide (0 μg, 150 μg, 100 μg, 75 μg, 50 μg, 25 and μg) first. EB (40 μL of a 2% solution was injected 10 min after injection of the peptide. Tissues (brains, lungs, heart, liver, spleen and kidney) were collected 1 h after EB delivery. All injections were through the femoral vein.

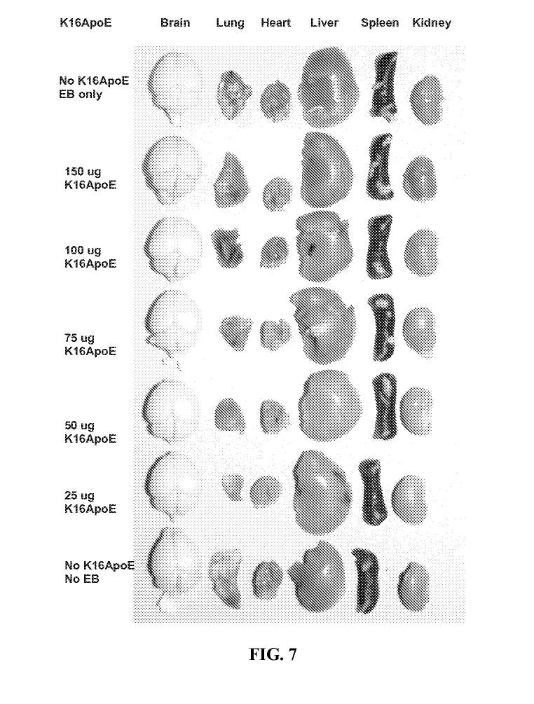

As shown in FIG. 7, it appears that: 1) EB goes into brain only after injection of the peptide (best with 150 μg of the peptide); 2) Kidney, spleen, liver and heart show some EB-stain when injected without K16ApoE; and 3) Kidney, spleen and liver are stained with EB when injected after K16ApoE injection. The results suggest the delivery of EB after K16ApoE injection is predominantly in organs known to express the LDLR (low-density lipoprotein receptor). Thus, delivery of EB in the brain (and in other organs) assisted by K16ApoE appears to be receptor-mediated.

### Example 8

### Administration of Cisplatin Across the BBB

For this experiment, different amount of K16ApoE was first injected via iv into the femoral vein, then different amounts of cisplatin was injected. Cardiac perfusion was done after 1 h followed by collection of brains. The brains were homogenized in 1% nitric acid, and the homogenate was centrifuged at 15,000 rpm in an Eppendorf microfuge for 10 min. The supernate was then collected and assayed for platinum content. Only ˜20% of the brains appeared to have dissolved in 1% nitric acid. The amount of platinum in the pellets (not dissolved in 1% nitric acid) has not been assayed at this time.

TABLE 1
Evaluation of delivery of cisplatin (cP) to the brain assisted by
K16ApoE.

| | Amount of cisplatin | | |
|---|---|---|---|
| | delivered to brain^{a} | | Fold-change^{b} |
| 300 μg cP alone | 9.46 | nanograms (ng) | |
| 300 μg cP + 300 μg | 739.14 | ng | 78.13 |
| K16ApoE | | | |
| 400 μg cP alone | 12.64 | ng | |
| 400 μg cP + 400 μg | 821.83 | ng | 65.02 |
| K16ApoE | | | |
| 500 μg cP alone | 16.32 | ng | |
| 500 μg cP + 500 μg | 1618.92 | ng | 99.20 |
| K16ApoE | | | |

^{a}cP was assayed in 1% nitric acid-soluble fraction of the brain, which comprised ~20% of brain tissue.
^{b}Fold-change is calculated as amount of cP delivered with K16ApoE divided by amount of cP delivered without the peptide.

Only one mouse in each category was used for this initial experiment.

### REFERENCES

1. Scheld W M. Drug delivery to the central nervous system: general principles and relevance to therapy for infections of the central nervous system. Rev Infect Dis. November-December; 11 Suppl 7:S1669-90. 1989.
2. Egleton R D, Davis T P. Bioavailability and transport of peptides and peptide drugs into the brain. Peptides. 18(9):1431-9. 1997.
3. van de Waterbeemd H, Camenisch G, Folkers G, Chretien J R, Raevsky O A. Estimation of blood-brain barrier crossing of drugs using molecular size and shape, and H-bonding descriptors. J Drug Target. 6(2):151-65. 1998.
4. Goldstein G W, Betz A L. The blood-brain barrier. Sci Am. 255(3):74-83. 1986.
5. Reese T S, Karnovsky M J. Fine structural localization of a blood-brain barrier to exogenous peroxidase. J Cell Biol. 34(1):207-17. 1967.
6. Reese T S, Karnovsky M J. Fine structural localization of a blood-brain barrier to exogenous peroxidase. J Cell Biol. 34(1):207-17. 1967.
7. Abbott N J, Rönnbäck L, Hansson E. Astrocyte-endothelial interactions at the bloodbrain barrier. Nat Rev Neurosci. (1):41-53. 2006.
8. Hdeib A, Sloan A E. Convection-enhanced delivery of 131I-chTNT-1/B mAB for treatment of high-grade adult gliomas. Expert Opin Biol Ther. 2011 June; 11(6):799-806. Epub 2011 Apr. 27.
9. Kioi M, Husain S R, Croteau D, Kunwar S, Puri R K. Convection-enhanced delivery of interleukin-13 receptor-directed cytotoxin for malignant glioma therapy. Technol Cancer Res Treat. 2006 June; 5(3):239-50.
10. Marquet F, Tung Y S, Teichert T, Ferrera V P, Konofagou E E. Noninvasive, transient and selective blood-brain barrier opening in non-human primates in vivo. PLoS One. 2011; 6(7):e22598.
11. Abraham M H. The factors that influence permeation across the blood-brain barrier. Eur J Med Chem. 2004 March; 39(3):235-40.
12. Blanchette M, Fortin D. Blood-brain barrier disruption in the treatment of brain tumors. Methods Mol Biol. 2011; 686:447-63.
13. Cosolo W C, Martinello P, Louis W J, Christophidis N. Blood-brain barrier disruption using mannitol: time course and electron microscopy studies. Am J Physiol. 1989 February; 256(2 Pt 2):R443-7.
14. Deng C X. Targeted drug delivery across the blood-brain barrier using ultrasound technique. Ther Deliv. 2010 Dec. 1; 1(6):819-848
15. Mesiwala A H, Farrell L, Wenzel H J, Silbergeld D L, Crum L A, Winn H R, Mourad P D. High-intensity focused ultrasound selectively disrupts the blood-brain barrier in vivo. Ultrasound Med Biol. 2002 March; 28(3):389-400.
16. Sheikov N, McDannold N, Vykhodtseva N, Jolesz F, Hynynen K. Cellular mechanisms of the blood-brain barrier opening induced by ultrasound in presence of microbubbles. Ultrasound Med Biol. 2004 July; 30(7):979-89.
17. Hynynen K, McDannold N, Vykhodtseva N, Raymond S, Weissleder R, Jolesz F A, Sheikov N. Focal disruption of the blood-brain barrier due to 260-kHz ultrasound bursts: a method for molecular imaging and targeted drug delivery. J Neurosurg. 2006 September; 105(3):445-54.
18. Choi J J, Feshitan J A, Baseri B, Wang S, Tung Y S, Borden M A, Konofagou E E. Microbubble-size dependence of focused ultrasound-induced blood-brain barrier opening in mice in vivo. IEEE Trans Biomed Eng. 2010 January; 57(1):145-54.
19. Waterhouse R N. Determination of lipophilicity and its use as a predictor of blood brain barrier penetration of molecular imaging agents. Mol Imaging Biol. 2003 November-December; 5(6):376-89.
20. Greig N H, Daly E M, Sweeney D J, Rapoport S I. Pharmacokinetics of chlorambuciltertiary butyl ester, a lipophilic chlorambucil derivative that achieves and maintains high concentrations in brain. Cancer Chemother Pharmacol. 1990; 25(5):320-5

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method of transporting an active agent across the blood-brain barrier of a patient, the method comprising:
(a) first administering to the patient an effective amount of a peptide comprising the sequence:
Xₙ-[B]ₘ
wherein:
X is a hydrophilic amino acid;
B is a blood-brain barrier agent;
n is an integer from 4 to 50; and
m is an integer from 1 to 3; and
(b) then administering to the patient an effective amount of the active agent.

2. A method of treating a brain disease in a patient, the method comprising:
(a) first administering to the patient an effective amount of a peptide comprising the sequence:
Xₙ-[B]ₘ
wherein:
X is a hydrophilic amino acid;
B is a blood-brain barrier agent;
n is an integer from 4 to 50; and
m is an integer from 1 to 3; and
(b) then administering to the patient a therapeutically effective amount of a biologically active agent for treatment of the brain disease.

3. A method of imaging the central-nervous system in a patient, the method comprising:
(a) first administering to the patient an effective amount of a peptide comprising the sequence:
Xₙ-[B]ₘ
wherein:
X is a hydrophilic amino acid;
B is a blood-brain barrier agent;
n is an integer from 4 to 50; and
m is an integer from 1 to 3; and
(b) then administering to the patient an effective amount of an imaging agent; and
(c) imaging the central-nervous system of the patient.

4. The method of claim 1, wherein each X is independently selected from the group consisting of arginine, asparagine, aspartic acid, glutamic acid, glutamine, histidine, lysine, serine, threonine, and tyrosine.

5. The method of claim 1, wherein each X is lysine.

6. The method of claim 1, wherein n is chosen from 4, 8, 12, 16, and 20.

7. The method of claim 6, wherein n is 16.

8. The method of claim 1, wherein m is 1.

9. The method of claim 1, wherein B is a receptor binding domain of an apolipoprotein.

10. The method of claim 9, wherein the receptor binding domain of an apolipoprotein is chosen from the receptor binding domain of ApoA, ApoB, ApoC, ApoD, ApoE, ApoE2, ApoE3, and ApoE4.

11. The method of claim 10, wherein the receptor binding domain of an apolipoprotein is chosen from the receptor binding domain of ApoB and ApoE.

12. The method of claim 1, wherein the active agent is a biologically active molecule or an imaging agent.

13-18. (canceled)

19. The method of claim 1, wherein the active agent is administered about 5 minutes to about 2 hours after the peptide.

20. The method of claim 1, wherein the peptide is administered in combination with a natural polypeptide capable of binding to a receptor at the blood-brain barrier.

21. The method of claim 20, wherein the natural polypeptide is IgG.

22. The method of claim 1, wherein the blood-brain barrier agent comprises a polypeptide sequence having at least 80% sequence identity to:
(SEQ ID NO: 13)
L-R-V-R-L-A-S-H-L-R-K-L-R-K-R-L-L-R-D-A;
(SEQ ID NO: 14)
S-S-V-I-D-A-L-Q-Y-K-L-E-G-T-T-R-L-T-R-K-R-G-L-K-L-
A-T-A-L-S-L-S-N-K-F-V-E-G-S-H;
(SEQ ID NO: 15)
Y-P-A-K-P-E-A-P-G-E-D-A-S-P-E-E-L-S-R-Y-Y-A-S-L-R-
H-Y-L-N-L-V-T-R-Q-R-Y*;
(SEQ ID NO: 16)
A-K-P-E-A-P-G-E-D-A-S-P-E-E-L-S-R-Y-Y-A-S-L-R-H-Y-
L-N-L-V-T-R-Q-R-Y*;
(SEQ ID NO: 17)
Y-P-S-D-P-D-N-P-G-E-D-A-P-A-E-D-L-A-R-Y-Y-S-A-L-R-
H-Y-I-N-L-I-T-R-Q-R-Y*;
(SEQ ID NO: 18)
A-P-L-E-P-V-Y-P-G-D-D-A-T-P-E-Q-M-A-Q-Y-A-A-E-L-R-
R-Y-I-N-M-L-T-R-P-R-Y*;
or
(SEQ ID NO: 19)
L-R-K-L-R-K-R-L-L-R-L-R-K-L-R-K-R-L-L-R,
wherein Y* is tyrosine or a tyrosine derivative.

23. (canceled)

24. (canceled)

25. The method of claim 22, wherein the peptide is less than 100 amino acids in length.

26. The method of claim 1, wherein the peptide is chosen from:
(SEQ ID NO: 39)
K-K-K-K-L-R-V-R-L-A-S-H-L-R-K-L-R-K-R-L-L-R-D-A;
(SEQ ID NO: 40)
K-K-K-K-K-K-K-K-L-R-V-R-L-A-S-H-L-R-K-L-R-K-R-L-L-
R-D-A;
(SEQ ID NO: 41)
K-K-K-K-K-K-K-K-K-K-K-K-L-R-V-R-L-A-S-H-L-R-K-L-R-
K-R-L-L-R-D-A;
(SEQ ID NO: 42)
K-K-K-K-K-K-K-K-K-K-K-K-K-K-K-K-L-R-V-R-L-A-S-H-L-
R-K-L-R-K-R-L-L-R-D-A;
(SEQ ID NO: 43)
K-K-K-K-K-K-K-K-K-K-K-K-K-K-K-K-K-K-K-K-L-R-V-R-L-
A-S-H-L-R-K-L-R-K-R-L-L-R-D-A;
(SEQ ID NO: 44)
K-K-K-K-S-S-V-I-D-A-L-Q-Y-K-L-E-G-T-T-R-L-T-R-K-R-
G-L-K-L-A-T-A-L-S-L-S-N-K-F-V-E-G-S-H;
(SEQ ID NO: 45)
K-K-K-K-K-K-K-K-S-S-V-I-D-A-L-Q-Y-K-L-E-G-T-T-R-L-
T-R-K-R-G-L-K-L-A-T-A-L-S-L-S-N-K-F-V-E-G-S-H;
(SEQ ID NO: 46)
K-K-K-K-K-K-K-K-K-K-K-K-S-S-V-I-D-A-L-Q-Y-K-L-E-G-
T-T-R-L-T-R-K-R-G-L-K-L-A-T-A-L-S-L-S-N-K-F-V-E-G-
S-H;
(SEQ ID NO: 47)
K-K-K-K-K-K-K-K-K-K-K-K-K-K-K-K-S-S-V-I-D-A-L-Q-Y-
K-L-E-G-T-T-R-L-T-R-K-R-G-L-K-L-A-T-A-L-S-L-S-N-K-
F-V-E-G-S-H;
and
(SEQ ID NO: 48)
K-K-K-K-K-K-K-K-K-K-K-K-K-K-K-K-K-K-K-K-S-S-V-I-D-
A-L-Q-Y-K-L-E-G-T-T-R-L-T-R-K-R-G-L-K-L-A-T-A-L-S-
L-S-N-K-F-V-E-G-S-H.

27. The method of claim 1, wherein the blood-brain barrier agent comprises the sequence:
(SEQ ID NO: 20)
L-R-X1-R-X2-X3-X4-H-L-R-X5-X6-X7-K-R-L-X8-R-D-X9
wherein:
X1 is selected from the group consisting of A, L, S, and V;
X2 is selected from the group consisting of L and M;
X3 is selected from the group consisting of A and S;
X4 is selected from the group consisting of N, S, and T;
X5 is selected from the group consisting of K and N;
X6 is selected from the group consisting of L, M, and V;
X7 is selected from the group consisting of R and P;
X8 is selected from the group consisting of L and M; and
X9 is selected from the group consisting of A and L.

28. The method of claim 27, wherein the blood-brain barrier agent is selected from the group consisting of:
(SEQ ID NO: 13)
L-R-V-R-L-A-S-H-L-R-K-L-R-K-R-L-L-R-D-A;
(SEQ ID NO: 21)
L-R-S-R-L-A-S-H-L-R-K-L-R-K-R-L-L-R-D-A;
(SEQ ID NO: 22)
L-R-V-R-M-A-S-H-L-R-K-L-R-K-R-L-L-R-D-A;
(SEQ ID NO: 23)
L-R-V-R-L-A-T-H-L-R-K-L-R-K-R-L-L-R-D-A;
(SEQ ID NO: 24)
L-R-V-R-L-A-S-H-L-R-K-L-P-K-R-L-L-R-D-A;
(SEQ ID NO: 25)
L-R-V-R-L-A-S-H-L-R-K-L-R-K-R-L-M-R-D-A;
(SEQ ID NO: 26)
L-R-V-R-L-A-S-H-L-R-N-L-R-K-R-L-L-R-D-A;
(SEQ ID NO: 27)
L-R-V-R-L-A-S-H-L-R-K-V-R-K-R-L-L-R-D-A;
(SEQ ID NO: 28)
L-R-V-R-M-S-S-H-L-R-K-L-R-K-R-L-L-R-D-A;
(SEQ ID NO: 29)
L-R-V-R-L-A-S-H-L-R-N-V-R-K-R-L-L-R-D-A;
(SEQ ID NO: 30)
L-R-V-R-L-A-S-H-L-R-N-M-R-K-R-L-L-R-D-A;
(SEQ ID NO: 31)
L-R-A-R-M-A-S-H-L-R-K-L-R-K-R-L-L-R-D-A;
(SEQ ID NO: 32)
L-R-V-R-L-S-S-H-L-R-K-L-R-K-R-L-M-R-D-A;
(SEQ ID NO: 33)
L-R-S-R-L-A-S-H-L-R-K-L-R-K-R-L-M-R-D-A;
(SEQ ID NO: 34)
L-R-V-R-L-S-S-H-L-P-K-L-R-K-R-L-L-R-D-A;
(SEQ ID NO: 35)
L-R-V-R-L-A-S-H-L-R-K-M-R-K-R-L-M-R-D-A;
(SEQ ID NO: 36)
L-R-V-R-L-A-S-H-L-R-N-L-P-K-R-L-L-R-D-A;
(SEQ ID NO: 37)
L-R-L-R-L-A-S-H-L-R-K-L-R-K-R-L-L-R-D-L;
and
(SEQ ID NO: 38)
L-R-V-R-L-A-N-H-L-R-K-L-R-K-R-L-L-R-D-L.

29. The method of claim 1, wherein the peptide comprises the sequence:
(SEQ ID NO: 141)
[X]ₙ-L-R-X1-R-X2-X3-X4-H-L-R-X5-X6-X7-K-R-L-X8-R-
D-X9
wherein:
X is a hydrophilic amino acid;
n is an integer from 4 to 50;
X1 is selected from the group consisting of A, L, S, and V;
X2 is selected from the group consisting of L and M;
X3 is selected from the group consisting of A and S;
X4 is selected from the group consisting of N, S, and T;
X5 is selected from the group consisting of K and N;
X6 is selected from the group consisting of L, M, and V;
X7 is selected from the group consisting of R and P;
X8 is selected from the group consisting of L and M; and
X9 is selected from the group consisting of A and L.

30. The method of claim 29, wherein the peptide is selected from the group consisting of:
(SEQ ID NO: 39)
K-K-K-K-L-R-V-R-L-A-S-H-L-R-K-L-R-K-R-L-L-R-D-A;
(SEQ ID NO: 40)
K-K-K-K-K-K-K-K-L-R-V-R-L-A-S-H-L-R-K-L-R-K-R-L-L-
R-D-A;
(SEQ ID NO: 41)
K-K-K-K-K-K-K-K-K-K-K-K-L-R-V-R-L-A-S-H-L-R-K-L-R-
K-R-L-L-R-D-A;
(SEQ ID NO: 42)
K-K-K-K-K-K-K-K-K-K-K-K-K-K-K-K-L-R-V-R-L-A-S-H-L-
R-K-L-R-K-R-L-L-R-D-A;
(SEQ ID NO: 43)
K-K-K-K-K-K-K-K-K-K-K-K-K-K-K-K-K-K-K-K-L-R-V-R-L-
A-S-H-L-R-K-L-R-K-R-L-L-R-D-A;
(SEQ ID NO: 49)
K-K-K-K-L-R-S-R-L-A-S-H-L-R-K-L-R-K-R-L-L-R-D-A;
(SEQ ID NO: 50)
K-K-K-K-K-K-K-K-L-R-S-R-L-A-S-H-L-R-K-L-R-K-R-L-L-
R-D-A;
(SEQ ID NO: 51)
K-K-K-K-K-K-K-K-K-K-K-K-L-R-S-R-L-A-S-H-L-R-K-L-R-
K-R-L-L-R-D-A;
(SEQ ID NO: 52)
K-K-K-K-K-K-K-K-K-K-K-K-K-K-K-K-L-R-S-R-L-A-S-H-L-
R-K-L-R-K-R-L-L-R-D-A;
(SEQ ID NO: 53)
K-K-K-K-K-K-K-K-K-K-K-K-K-K-K-K-K-K-K-K-L-R-S-R-L-
A-S-H-L-R-K-L-R-K-R-L-L-R-D-A;
(SEQ ID NO: 54)
K-K-K-K-L-R-V-R-M-A-S-H-L-R-K-L-R-K-R-L-L-R-D-A;
(SEQ ID NO: 55)
K-K-K-K-K-K-K-K-L-R-V-R-M-A-S-H-L-R-K-L-R-K-R-L-L-
R-D-A;
(SEQ ID NO: 56)
K-K-K-K-K-K-K-K-K-K-K-K-L-R-V-R-M-A-S-H-L-R-K-L-R-
K-R-L-L-R-D-A;
(SEQ ID NO: 57)
K-K-K-K-K-K-K-K-K-K-K-K-K-K-K-K-L-R-V-R-M-A-S-H-L-
R-K-L-R-K-R-L-L-R-D-A;
(SEQ ID NO: 58)
K-K-K-K-K-K-K-K-K-K-K-K-K-K-K-K-K-K-K-K-L-R-V-R-M-
A-S-H-L-R-K-L-R-K-R-L-L-R-D-A;
(SEQ ID NO: 59)
K-K-K-K-L-R-V-R-L-A-T-H-L-R-K-L-R-K-R-L-L-R-D-A;
(SEQ ID NO: 60)
K-K-K-K-K-K-K-K-L-R-V-R-L-A-T-H-L-R-K-L-R-K-R-L-L-
R-D-A;
(SEQ ID NO: 61)
K-K-K-K-K-K-K-K-K-K-K-K-L-R-V-R-L-A-T-H-L-R-K-L-R-
K-R-L-L-R-D-A;
(SEQ ID NO: 62)
K-K-K-K-K-K-K-K-K-K-K-K-K-K-K-K-L-R-V-R-L-A-T-H-L-
R-K-L-R-K-R-L-L-R-D-A;
(SEQ ID NO: 63)
K-K-K-K-K-K-K-K-K-K-K-K-K-K-K-K-K-K-K-K-L-R-V-R-L-
A-T-H-L-R-K-L-R-K-R-L-L-R-D-A;
(SEQ ID NO: 64)
K-K-K-K-L-R-V-R-L-A-S-H-L-R-K-L-P-K-R-L-L-R-D-A;
(SEQ ID NO: 65)
K-K-K-K-K-K-K-K-L-R-V-R-L-A-S-H-L-R-K-L-P-K-R-L-L-
R-D-A;
(SEQ ID NO: 66)
K-K-K-K-K-K-K-K-K-K-K-K-L-R-V-R-L-A-S-H-L-R-K-L-P-
K-R-L-L-R-D-A;
(SEQ ID NO: 67)
K-K-K-K-K-K-K-K-K-K-K-K-K-K-K-K-L-R-V-R-L-A-S-H-L-
R-K-L-P-K-R-L-L-R-D-A;
(SEQ ID NO: 68)
K-K-K-K-K-K-K-K-K-K-K-K-K-K-K-K-K-K-K-K-L-R-V-R-L-
A-S-H-L-R-K-L-P-K-R-L-L-R-D-A;
(SEQ ID NO: 69)
K-K-K-K-L-R-V-R-L-A-S-H-L-R-K-L-R-K-R-L-M-R-D-A;
(SEQ ID NO: 70)
K-K-K-K-K-K-K-K-L-R-V-R-L-A-S-H-L-R-K-L-R-K-R-L-M-
R-D-A;
(SEQ ID NO: 71)
K-K-K-K-K-K-K-K-K-K-K-K-L-R-V-R-L-A-S-H-L-R-K-L-R-
K-R-L-M-R-D-A;
(SEQ ID NO: 72)
K-K-K-K-K-K-K-K-K-K-K-K-K-K-K-K-L-R-V-R-L-A-S-H-L-
R-K-L-R-K-R-L-M-R-D-A;
(SEQ ID NO: 73)
K-K-K-K-K-K-K-K-K-K-K-K-K-K-K-K-K-K-K-K-L-R-V-R-L-
A-S-H-L-R-K-L-R-K-R-L-M-R-D-A;
(SEQ ID NO: 74)
K-K-K-K-L-R-V-R-L-A-S-H-L-R-N-L-R-K-R-L-L-R-D-A;
(SEQ ID NO: 75)
K-K-K-K-K-K-K-K-L-R-V-R-L-A-S-H-L-R-N-L-R-K-R-L-L-
R-D-A;
(SEQ ID NO: 76)
K-K-K-K-K-K-K-K-K-K-K-K-L-R-V-R-L-A-S-H-L-R-N-L-R-
K-R-L-L-R-D-A;
(SEQ ID NO: 77)
K-K-K-K-K-K-K-K-K-K-K-K-K-K-K-K-L-R-V-R-L-A-S-H-L-
R-N-L-R-K-R-L-L-R-D-A;
(SEQ ID NO: 78)
K-K-K-K-K-K-K-K-K-K-K-K-K-K-K-K-K-K-K-K-L-R-V-R-L-
A-S-H-L-R-N-L-R-K-R-L-L-R-D-A;
(SEQ ID NO: 79)
K-K-K-K-L-R-V-R-L-A-S-H-L-R-K-V-R-K-R-L-L-R-D-A;
(SEQ ID NO: 80)
K-K-K-K-K-K-K-K-L-R-V-R-L-A-S-H-L-R-K-V-R-K-R-L-L-
R-D-A;
(SEQ ID NO: 81)
K-K-K-K-K-K-K-K-K-K-K-K-L-R-V-R-L-A-S-H-L-R-K-V-R-
K-R-L-L-R-D-A;
(SEQ ID NO: 82)
K-K-K-K-K-K-K-K-K-K-K-K-K-K-K-K-L-R-V-R-L-A-S-H-L-
R-K-V-R-K-R-L-L-R-D-A;
(SEQ ID NO: 83)
K-K-K-K-K-K-K-K-K-K-K-K-K-K-K-K-K-K-K-K-L-R-V-R-L-
A-S-H-L-R-K-V-R-K-R-L-L-R-D-A;
(SEQ ID NO: 84)
K-K-K-K-L-R-V-R-M-S-S-H-L-R-K-L-R-K-R-L-L-R-D-A;
(SEQ ID NO: 85)
K-K-K-K-K-K-K-K-L-R-V-R-M-S-S-H-L-R-K-L-R-K-R-L-L-
R-D-A;
(SEQ ID NO: 86)
K-K-K-K-K-K-K-K-K-K-K-K-L-R-V-R-M-S-S-H-L-R-K-L-R-
K-R-L-L-R-D-A;
(SEQ ID NO: 87)
K-K-K-K-K-K-K-K-K-K-K-K-K-K-K-K-L-R-V-R-M-S-S-H-L-
R-K-L-R-K-R-L-L-R-D-A;
(SEQ ID NO: 88)
K-K-K-K-K-K-K-K-K-K-K-K-K-K-K-K-K-K-K-K-L-R-V-R-M-
S-S-H-L-R-K-L-R-K-R-L-L-R-D-A;
(SEQ ID NO: 89)
K-K-K-K-L-R-V-R-L-A-S-H-L-R-N-V-R-K-R-L-L-R-D-A;
(SEQ ID NO: 90)
K-K-K-K-K-K-K-K-L-R-V-R-L-A-S-H-L-R-N-V-R-K-R-L-L-
R-D-A;
(SEQ ID NO: 91)
K-K-K-K-K-K-K-K-K-K-K-K-L-R-V-R-L-A-S-H-L-R-N-V-R-
K-R-L-L-R-D-A;
(SEQ ID NO: 92)
K-K-K-K-K-K-K-K-K-K-K-K-K-K-K-K-L-R-V-R-L-A-S-H-L-
R-N-V-R-K-R-L-L-R-D-A;
(SEQ ID NO: 93)
K-K-K-K-K-K-K-K-K-K-K-K-K-K-K-K-K-K-K-K-L-R-V-R-L-
A-S-H-L-R-N-V-R-K-R-L-L-R-D-A;
(SEQ ID NO: 94)
K-K-K-K-L-R-V-R-L-A-S-H-L-R-N-M-R-K-R-L-L-R-D-A;
(SEQ ID NO: 95)
K-K-K-K-K-K-K-K-L-R-V-R-L-A-S-H-L-R-N-M-R-K-R-L-L-
R-D-A;
(SEQ ID NO: 96)
K-K-K-K-K-K-K-K-K-K-K-K-L-R-V-R-L-A-S-H-L-R-N-M-R-
K-R-L-L-R-D-A;
(SEQ ID NO: 97)
K-K-K-K-K-K-K-K-K-K-K-K-K-K-K-K-L-R-V-R-L-A-S-H-L-
R-N-M-R-K-R-L-L-R-D-A;
(SEQ ID NO: 98)
K-K-K-K-K-K-K-K-K-K-K-K-K-K-K-K-K-K-K-K-L-R-V-R-L-
A-S-H-L-R-N-M-R-K-R-L-L-R-D-A;
(SEQ ID NO: 99)
K-K-K-K-L-R-A-R-M-A-S-H-L-R-K-L-R-K-R-L-L-R-D-A;
(SEQ ID NO: 100)
K-K-K-K-K-K-K-K-L-R-A-R-M-A-S-H-L-R-K-L-R-K-R-L-L-
R-D-A;
(SEQ ID NO: 101)
K-K-K-K-K-K-K-K-K-K-K-K-L-R-A-R-M-A-S-H-L-R-K-L-R-
K-R-L-L-R-D-A;
(SEQ ID NO: 102)
K-K-K-K-K-K-K-K-K-K-K-K-K-K-K-K-L-R-A-R-M-A-S-H-L-
R-K-L-R-K-R-L-L-R-D-A;
(SEQ ID NO: 103)
K-K-K-K-K-K-K-K-K-K-K-K-K-K-K-K-K-K-K-K-L-R-A-R-M-
A-S-H-L-R-K-L-R-K-R-L-L-R-D-A;
(SEQ ID NO: 104)
K-K-K-K-L-R-V-R-L-S-S-H-L-R-K-L-R-K-R-L-M-R-D-A;
(SEQ ID NO: 105)
K-K-K-K-K-K-K-K-L-R-V-R-L-S-S-H-L-R-K-L-R-K-R-L-M-
R-D-A;
(SEQ ID NO: 106)
K-K-K-K-K-K-K-K-K-K-K-K-L-R-V-R-L-S-S-H-L-R-K-L-R-
K-R-L-M-R-D-A;
(SEQ ID NO: 107)
K-K-K-K-K-K-K-K-K-K-K-K-K-K-K-K-L-R-V-R-L-S-S-H-L-
R-K-L-R-K-R-L-M-R-D-A;
(SEQ ID NO: 108)
K-K-K-K-K-K-K-K-K-K-K-K-K-K-K-K-K-K-K-K-L-R-V-R-L-
S-S-H-L-R-K-L-R-K-R-L-M-R-D-A;
(SEQ ID NO: 109)
K-K-K-K-L-R-S-R-L-A-S-H-L-R-K-L-R-K-R-L-M-R-D-A;
(SEQ ID NO: 110)
K-K-K-K-K-K-K-K-L-R-S-R-L-A-S-H-L-R-K-L-R-K-R-L-M-
R-D-A;
(SEQ ID NO: 111)
K-K-K-K-K-K-K-K-K-K-K-K-L-R-S-R-L-A-S-H-L-R-K-L-R-
K-R-L-M-R-D-A;
(SEQ ID NO: 112)
K-K-K-K-K-K-K-K-K-K-K-K-K-K-K-K-L-R-S-R-L-A-S-H-L-
R-K-L-R-K-R-L-M-R-D-A;
(SEQ ID NO: 113)
K-K-K-K-K-K-K-K-K-K-K-K-K-K-K-K-K-K-K-K-L-R-S-R-L-
A-S-H-L-R-K-L-R-K-R-L-M-R-D-A;
(SEQ ID NO: 114)
K-K-K-K-L-R-V-R-L-S-S-H-L-P-K-L-R-K-R-L-L-R-D-A;
(SEQ ID NO: 115)
K-K-K-K-K-K-K-K-L-R-V-R-L-S-S-H-L-P-K-L-R-K-R-L-L-
R-D-A;
(SEQ ID NO: 116)
K-K-K-K-K-K-K-K-K-K-K-K-L-R-V-R-L-S-S-H-L-P-K-L-R-
K-R-L-L-R-D-A;
(SEQ ID NO: 117)
K-K-K-K-K-K-K-K-K-K-K-K-K-K-K-K-L-R-V-R-L-S-S-H-L-
P-K-L-R-K-R-L-L-R-D-A;
(SEQ ID NO: 118)
K-K-K-K-K-K-K-K-K-K-K-K-K-K-K-K-K-K-K-K-L-R-V-R-L-
S-S-H-L-P-K-L-R-K-R-L-L-R-D-A;
(SEQ ID NO: 119)
K-K-K-K-L-R-V-R-L-A-S-H-L-R-K-M-R-K-R-L-M-R-D-A;
(SEQ ID NO: 120)
K-K-K-K-K-K-K-K-L-R-V-R-L-A-S-H-L-R-K-M-R-K-R-L-M-
R-D-A;
(SEQ ID NO: 121)
K-K-K-K-K-K-K-K-K-K-K-K-L-R-V-R-L-A-S-H-L-R-K-M-R-
K-R-L-M-R-D-A;
(SEQ ID NO: 122)
K-K-K-K-K-K-K-K-K-K-K-K-K-K-K-K-L-R-V-R-L-A-S-H-L-
R-K-M-R-K-R-L-M-R-D-A;
(SEQ ID NO: 123)
K-K-K-K-K-K-K-K-K-K-K-K-K-K-K-K-K-K-K-K-L-R-V-R-L-
A-S-H-L-R-K-M-R-K-R-L-M-R-D-A;
(SEQ ID NO: 124)
K-K-K-K-L-R-V-R-L-A-S-H-L-R-N-L-P-K-R-L-L-R-D-A;
(SEQ ID NO: 125)
K-K-K-K-K-K-K-K-L-R-V-R-L-A-S-H-L-R-N-L-P-K-R-L-L-
R-D-A;
(SEQ ID NO: 126)
K-K-K-K-K-K-K-K-K-K-K-K-L-R-V-R-L-A-S-H-L-R-N-L-P-
K-R-L-L-R-D-A;
(SEQ ID NO: 127)
K-K-K-K-K-K-K-K-K-K-K-K-K-K-K-K-L-R-V-R-L-A-S-H-L-
R-N-L-P-K-R-L-L-R-D-A;
(SEQ ID NO: 128)
K-K-K-K-K-K-K-K-K-K-K-K-K-K-K-K-K-K-K-K-L-R-V-R-L-
A-S-H-L-R-N-L-P-K-R-L-L-R-D-A;
(SEQ ID NO: 129)
K-K-K-K-L-R-L-R-L-A-S-H-L-R-K-L-R-K-R-L-L-R-D-L;
(SEQ ID NO: 130)
K-K-K-K-K-K-K-K-L-R-L-R-L-A-S-H-L-R-K-L-R-K-R-L-L-
R-D-L;
(SEQ ID NO: 131)
K-K-K-K-K-K-K-K-K-K-K-K-L-R-L-R-L-A-S-H-L-R-K-L-R-
K-R-L-L-R-D-L;
(SEQ ID NO: 132)
K-K-K-K-K-K-K-K-K-K-K-K-K-K-K-K-L-R-L-R-L-A-S-H-L-
R-K-L-R-K-R-L-L-R-D-L;
(SEQ ID NO: 133)
K-K-K-K-K-K-K-K-K-K-K-K-K-K-K-K-K-K-K-K-L-R-L-R-L-
A-S-H-L-R-K-L-R-K-R-L-L-R-D-L;
(SEQ ID NO: 134)
K-K-K-K-L-R-V-R-L-A-N-H-L-R-K-L-R-K-R-L-L-R-D-L;
(SEQ ID NO: 135)
K-K-K-K-K-K-K-K-L-R-V-R-L-A-N-H-L-R-K-L-R-K-R-L-L-
R-D-L;
(SEQ ID NO: 136)
K-K-K-K-K-K-K-K-K-K-K-K-L-R-V-R-L-A-N-H-L-R-K-L-R-
K-R-L-L-R-D-L;
(SEQ ID NO: 137)
K-K-K-K-K-K-K-K-K-K-K-K-K-K-K-K-L-R-V-R-L-A-N-H-L-
R-K-L-R-K-R-L-L-R-D-L;
and
(SEQ ID NO: 138)
K-K-K-K-K-K-K-K-K-K-K-K-K-K-K-K-K-K-K-K-L-R-V-R-L-
A-N-H-L-R-K-L-R-K-R-L-L-R-D-L.
